SEQUENCE LISTING (5 sequences)

SEQ ID NO: 1 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 2 - protein, 216 aa, Homo sapiens
LHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 3 - protein, 5 aa, Artificial
  synthetic
GSGGS

SEQ ID NO: 4 - protein, 5 aa, Artificial
  synthetic
GGGGS

SEQ ID NO: 5 - protein, 4 aa, Artificial
  synthetic
GGGS

(19) United States
(12) Patent Application Publication
(10) Pub. No.: US 2005/0181994 A1
(43) Pub. Date: Aug. 18, 2005

(54) Novel variants of CD40L protein

(75) Inventors: Aaron Keith Chamberlain, Pasadena, CA (US); John R. Desjarlais, Pasadena, CA (US); Gregory L. Moore, Pasadena, CA (US)

Correspondence Address: Robin M. Silva;Dorsey & Whitney LLP, Intellectual Property Department, Four Embarcadero Center, Suite 3400, San Francisco, CA, 94111-4187, US

(73) Assignee: Xencor, Inc.

(21) Appl. No.: 10/952,493

(22) Filed: Oct. 12, 2004

Related U.S. Application Data
(63) Continuation-in-part of application No. 10/338,083, filed on Jan. 6, 2003.
(63) Continuation-in-part of application No. 10/338,785, filed on Jan. 6, 2003.
(60) Provisional application No. 60/510,430, filed on Oct. 10, 2003.
(60) Provisional application No. 60/517,728, filed on Nov. 5, 2003.
(60) Provisional application No. 60/523,545, filed on Nov. 20, 2003.

(51) Int. Cl. A61K 38/17; C07K 14/74

(52) U.S. Cl. 514/12; 530/350

(57) ABSTRACT

The present invention relates to soluble, recombinant CD40L variant proteins that may be expressed solubly in E. coli. The variants of the present invention may substantially reduced binding to alpha IIb-beta3 integrin, act as CD40L antagonists or agonists, and methods for generating the same.

This application claims benefit of priority under 35 U.S.C. §119(e) of 60/510,430, filed Oct. 10, 2003, U.S. Ser. No. 60/517,728, filed Nov. 5, 2003, and 60/523,545, filed Nov. 20, 2003, all of which are hereby incorporated by reference in their entirety. In addition, the application is a continuation in part application of both U.S. Ser. No. 10/338,785, filed Jan. 6, 2003 and its continuation-in-part, filed Jul. 7, 2003, both of which are expressly incorporated by reference in their entirety.

### FIELD OF THE INVENTION

The present invention relates to novel variants of the extracellular domains (ECD) of human CD40L proteins and fragments, derivatives, conformers, and analogs thereof. These novel variants comprise CD40L variants that antagonize wild-type CD40L, do not bind to the integrin alphaIIbbeta3, and CD40L variants that act as superagonists.

### BACKGROUND OF THE INVENTION

CD40L, also known as CD154, TNFSF5, TRAP, and gp39, is a member of the TNF superfamily which may trimerize to bind and activate CD40, as well as alpha IIb-beta3 integrin. CD40L is about 30-kDa, the full-length version has 261 amino acids of which the Extra Cellular Domain (ECD) is amino acids 45-261). It is a type II membrane glycoprotein. In some physiological contexts, CD40L is processed to yield a soluble form comprised of amino acids 113-261. Elevated levels of this soluble form have been established for a variety of disease conditions, including but not limited to: chronic renal failure, diabetes, inflammatory bowel disease, autoimmune thrombocytopenic purpura, Hodgkin's disease, rheumatoid vasculitis, systemic lupus erythrematosis, chronic lymphocytic leukaemia, preeclampsia, sickle cell anemia, atherosclerosis, and numerous cardiovascular conditions. Elevated levels of soluble CD40L have also become well established as a reliable predictor of cardiovascular events.

CD40L is transiently expressed after MHC/peptide-induced TCR activation on CD4+ T cells. It is contact-dependent, and is essential for effector signaling between T and B cells. Mutations in CD40L cause X-linked hyper IgM syndrome, which causes severe Immunodeficiency, low levels of IgA, IgG, and IgE, and affected patients cannot mount a thymus-dependent humoral response. See, Sacco, et al, Cancer Gene Therapy, 7(10): 1299-1306 (2002) and Kuniaki et al, J Biol. Chem, 274(16): 11310-11320 (1999). The pleiotropic immunological effects of CD40-CD40L interactions include autoimmunity, transplantation and allograft rejection, as well as control of infection.

CD40L has many potential therapeutic indications, anti-tumor or oncological conditions, including Hodgkins and non-Hodgkins Lymphomas (NHL), pre- and post-transplantation immunosuppression, psoriasis, rheumatoid and collagen-induced arthritis, multiple sclerosis, systemic lupus erythematosus (SLE), allergic encephalitis, acute and chronic graft versus host disease, Crohn's disease, diabetes, chronic renal failure, mixed connective tissue disease, sickle cell anemia, inflammatory bowel disease, Hodgkin disease, rheumatoid vasculitis, chronic lymphocytic leukaemia, preeclampsia and cardiovascular conditions including atherosclerosis, thrombocytopenia (Purpura), etc. Table 1 below obtained from Cancer Gene Therapy (2003) 10, 1-13 shows the in vitro outcome of CD40 receptor binding on normal cells. The outcomes provide for a rich source of potential therapeutic indications.

TABLE 1
In vitro outcome of CD40 receptor binding on normal cells

| Cell type | Outcome |
|---|---|
| Resting B cells | Isotype switching maturation to memory B cell or plasma cell; cytokine secretion, CD23, VLA-4, CD80/86 |
| | up-regulation |
| Activated B cells | Growth inhibition and increased apoptosis, increased Fas/Apo-1 expression |
| Monocyte/macrophages | Decreased apoptosis; up-regulated expression of CD54, CD86, CD40, MHC Class II; Increased cytokine secreation |
| | (IL-1β, IL-6, IL-8, IL-12, TNFα, IFNγ) and nitric oxide production |
| Monocytic dendritic cells | Increased survival; cytokine (IL-1, IL-6, IL-8, IL-10, IL-12, TNFα, MIP-1α) and enzyme (MMP) secretion; |
| | up-regulation of costimulatory molecules (CD54/ICAM-1, CD58/LFA-3, CD80/B7-1, CD86/B7-2) |
| Follicular dendritic cells | Growth stimulation |
| T cells | Proliferation; IL-2 receptor up-regulation |
| Endothelial cells | Activation, up-regulation of CD54/ICAM-1, CD106/VCAM-1, CD62E/E-selectin, enhancing leukocyte adherence |
| Basal epithelium | Cytostatic/growth inhibition |
| Thymic epithelial cells | GM-CSF secretion, growth inhibition |
| Kidney eptihelial cells | RANTES secretion |
| Keratinocytes | Growth inhibition; IL-6, IL-8, TNFα secretion; CD54 up-regulation |
| Fibroblasts | IL-6 production, growth stimulation |

VLA: very late antigen; TNF: tumor necrosis factor; IFN: interferon; MIP: macrophage inflammatory protein; MMP: matrix metalloproteinase; ICAM: Intracellular adhesion molecule; LFA: leukocyte functional antigen; VCAM: vascular cell adhesion molecule; GM-CSF: granulocyte-macrophage colony-stimulating factor; RANTES: regulated on activation, normal T cell expressed and secreted.

However, the clinical experience with CD40L (including monoclonal antibodies) has not yet produced an effective therapeutic. See, Curr Opin Investig Drugs 2002 May; 3(5):725-34, discussing monoclonal antibody IDEC-131; also the Biogen/Columbia University monoclonal antibody ruplizumab (anti-CD40L) Phase II Antova trial was discontinued due to thrombo-embolic adverse effects. Recently, evidence has accumulated indicating that CD40L can activate platelets by signaling through alpha IIb-beta3 integrin (see for example Prasad et al., Proc Natl Acad Sci U S A. 2003 Oct. 14; 100(21):12367-71).

Accordingly, a need exists for CD40L antagonists and superagonists that do not bind alpha IIb-beta3 integrin. These variants of CD40L will have improved therapeutic efficacy and safety, as they substantially reduce or eliminate immune cell activation through CD40 signaling and will have reduced risk of platelet activation through alpha IIb-beta3 integrin signaling.

### SUMMARY OF THE INVENTION

The present invention is directed at generating novel variants of human CD40L protein, comprising the TNF homology domain of CD40L, which behave as CD40L antagonists or superagonists. CD40L antagonists may comprise dominant-negative CD40L proteins and/or competitive inhibitors of CD40L. An additional aspect of the invention is the identification of modifications that confer reduced binding to alpha IIb-beta3 integrin as compared to wild-type CD40L. The reduction in binding to alpha IIb-beta3 integrin is important for the development of a more safe and effective therapeutic, as these novel CD40L proteins avoid platelet activation.

An aspect of the present invention is non-naturally occurring CD40L variants that express solubly in bacteria, including but not limited to E. coli. In previous studies, it has been observed that human CD40L forms inclusion bodies when expressed in E. coli. Alternate production routes such as refolding from inclusion bodies or mammalian expression are significantly more expensive and time consuming than soluble bacterial expression. Soluble bacterial expression facilitates the discovery, characterization, and production of novel CD40L variants. It is a further object of the invention to provide a method that can be used to engineer variants of other proteins that express solubly in bacteria, including but not limited to E. coli.

The invention further relates to the design of human CD40L antagonists that inhibit the interaction between CD40L receptors and CD40L and methods for generating the same.

An aspect of the present invention is non-naturally occurring CD40L variants that: 1) do not appreciably agonize CD40 activity; 2) antagonize CD40 activity; 3) exchange with wild-type CD40L (that is, form trimers containing at least one wild-type CD40L protein monomer and at least one variant CD40L protein monomer), and 4) interfere with or avoid CD40L-mediated platelet activation, thrombi formation and/or fibrinogen binding. Such variants are referred to as “dominant negative” variants.

A further object of the present invention is CD40L variants that preferentially hetero-oligomerize, and more specifically hetero-trimerize, with wild-type CD40L.

An aspect of the present invention is CD40L variants that: 1) do not appreciably agonize CD40 activity; 2) antagonize CD40 activity; 3) compete with wild-type CD40L for binding the CD40L receptors, and 4) interfere with or avoid CD40L-mediated platelet activation, thrombi formation and/or fibrinogen binding. Such variants are referred to as “competitive inhibitor” variants.

A further object of the present invention is CD40L variants that have significantly less ability to activate platelets.

A further object of the present invention is CD40L variants that have significantly less ability to bind and/or activate alpha IIb-beta3 integrin.

The invention further relates to the design of human CD40L superagonists that bind and activate the CD40L receptors more strongly than the wild-type human CD40L protein does.

In a further aspect, the invention provides recombinant nucleic acids encoding the variant CD40L proteins, expression vectors, and host cells.

In an additional aspect, the invention provides methods of producing a variant CD40L protein comprising culturing the host cells of the invention under conditions suitable for expression of the variant CD40L protein.

In a further aspect, the invention provides pharmaceutical compositions comprising a variant CD40L protein of the invention and a pharmaceutical carrier.

In a further aspect, the invention provides methods for treating CD40L-related disorders comprising administering a variant CD40L protein of the invention to a patient.

In an additional aspect, the invention provides CD40L variants that exchange with and attenuate the signaling potency of soluble CD40L.

In a further aspect, the invention provides variant CD40L monomer proteins, as compared to the human CD40L sequence, comprising a variant of an extracellular domain of a wild-type CD40L protein. The extracellular domain is from amino acids 45-261 (using the numbering of the figures), but as will be appreciated by those in the art, positions 45-120 or 121 are optional.

In an additional aspect, the invention provides variant CD40L proteins wherein the variant is a substitution of a cysteine residue to a non-cysteine residue.

In an additional aspect, the variant CD40L proteins of the invention have at least one substitution, insertion or deletion (as compared to human wild-type CD40L) of a position selected from the group consisting of 121, 123, 125, 140, 141, 142, 143, 144, 145, 146, 148, 166, 167, 168, 170, 172, 174, 176, 177, 178, 179, 181, 182, 185, 186, 187, 188, 189, 190, 190, 191, 192, 197, 198, 199, 200, 201, 203, 204, 205, 206, 207, 208, 209, 210, 211, 214, 215, 216, 217, 218, 219, 220, 221, 222, 223, 224, 225, 226, 227, 228, 229, 230, 242, 243, 244, 245, 246, 247, 248, 249, 250, 251, 252, 253, 255, 259, 260 and 261 (FIG. 2 TNFA corresponding positions 11, 13, 15, 28, 29, 30, 31, 32, 33, 34, 36, 53, 54, 55, 57, 59, 61, 63, 64, 65, 66, 68, 69, 72, 73, 74, 75, 76, 77, 78, 79, 84, 85, 86, 87, 88, 89, 91, 92, 93, 94, 95, 96, 97, 98, 99, 102, 103, 104, 109, 112, 113, 114, 115, 116, 117, 118, 119, 120, 121, 122, 123, 124, 125, 137, 138, 139, 140, 141, 142, 143, 144, 145, 146 147, 148, 149, 151, 155, 156 and 157). 1, 2, 3, 4 and 5 substitutions, insertions and deletions are preferred, with the remaining of the protein generally being the human sequence. One preferred subset has at least one variant position selected from the group consisting of: 127, 130, 142, 143, 144, 145, 146, 176, 178, 184, 185, 186, 187, 188, 200, 201, 203, 207, 209, 218, 220, 242, 245, 248, 249, 251, 253. Another preferred subset has one or more variant positions selected from the group consisting of: 120, 127, 134, 137, 145, 146, 148, 152, 155, 178, 183, 188, 190, 198, 201, 204, 217, 218, 233, and 261. Another preferred subset has one or more variant positions selected from the group consisting of 36, 96, 123, 125, 126, 128, 129, 140, 142, 143, 144, 145, 147, 155, 169, 170, 173, 174, 176, 183, 184, 186, 193, 195, 200, 202, 203, 208, 218, 220, 221, 224, 226, 227, 231, 232, 235, 236, 237, 254, 257, 257, and 258. An additional preferred subset has one or more variant positions selected from the group consisting of 36, 96, 123, 125, 126, 128, 129, 140, 142, 143, 144, 145, 147, 155, 169, 170, 173, 174, 176, 183, 184, 186, 193, 195, 200, 202, 203, 208, 218, 220, 221, 224, 226, 227, 231, 232, 235, 236, 237, 254, 257, 257, and 258.

Thus, the invention provides variant CD40L proteins wherein at least one CD40L large domain receptor contact position is substituted at the following positions: I127, A130, E142, K143, G144, Y145, Y146, T176, C178, C218, Q220, S245, S248, H249, T251, and F253. Small domain receptor contact positions are substituted at the following positions: S185, Q186, A187, P188, I190, R207, A209, S214, and T242. CD40L DE loop receptor contact positions are substituted at the following positions: R200, F201, and R203.

In a further aspect, variant CD40L proteins (again, as for most references herein, and as outlined below, the proteins can be monomers, dimers or trimers,) wherein at least one substitution is selected from the group consisting of: I127H, I127Q, I127E, I127C, A130P, A130Q, A130K, A130M, A130N, E142M, E142V, E142C, E142I, G144Q, G144M, G144E, G144H, Y145P, Y145E, Y145N, Y146S, Y146C, T176G, C178G, C178S, C178D, C178E, C178H, C178Q, C178N, C178K, C178Y, C178M, S184C, S184L, S184R, S184M, S185R, S185H, Q186T, Q1861, Q186V, Q186L, Q186A, Q186C, A187E, A187H, A187W, A187Q, A187M, A187D, A187L, P188D, P188N, P188H, P188E, P188M, P188C, P188Q, P188K, P188T, P188A, P188V, P188S, I190Y, I190A, I190C, I190S, I190E, R200D, R200M, R200S, F201R, F201H, F201E, F201N, F201Q, F201D, F201K, F201S, F201T, R203Y, R203F, R203T, R203W, R203A, R203SS, R203D, R207C, S214P, C218P, Q220P, Q220C, Q220T, S245R, S245P, S245H, S245M, S245V, S245L, S245Y, S2451, S248C, H249D, T251 E, T251Q, T251M, T251D, T251P, T251H, T251I, T251L, F253E, F253D, F253H.

An additional aspect has one or more modifications (e.g. substitutions) selected from the group consisting of: I127Q, I127E, I127S, I127D, A130Q, A130K, A130N, A130R, A130D, A130E, G144Q, G144E, G144K, G144R, Y145E, Y145N, Y145D, Y146S, Y146N, C178S, C178D, C178E, C178Q, C178N, C178K, C178R, S184R, S185R, A187E, A187Q, A187D, A187N, A187K, A187R, P188D, P188N, P188E, P188Q, P188K, P188S, I190S, I190E, R200D, R200S, F201R, F201E, F201N, F201Q, F201D, F201K, F201S, R203S, R203D, S214R, C218D, S245R, H249D, T251E, T251Q, T251D, T251R, F253E, and F253D.

A further aspect has one or more modifications (e.g. substitutions) selected from the group of substitutions consisting of: P120R, P120Q, P120N, P120E, P120D, P120K, P120G, P120S, I127Q, I127E, I127S, I127D, T134Q, T134E, T134D, T134R, T134N, T134K, T134S, T134G, V137R, V137K, V137S, Y145E, Y145N, Y145D, Y146S, Y146N, M148K, L152E, L152Q, C178G, C178S, C178D, C178E, C178Q, C178N, C178K, C178R, A183Q, A183E, A183R, A183D, A183N, A183K, A183S, A183G, P188D, P188N, P188E, P188Q, P188K, P188S, P188G, I190S, I190E, P198E, P198D, P198N, P198Q, F201R, F201E, F201N, F201Q, F201D, F201K, F201S, F201G, I204E, I204Q, I204S, P217D, P217E, P217Q, P217S, P217N, P217G, C218G, C218D, P233E, P233D, P233S, P233Q, P233N, P233R, P233G, L261N, L261S, and L261D.

An additional aspect has one or more modifications (e.g. substitutions) selected from the group consisting of: M36R, K96N, A123E, H125R, V126D, V126A, S128R, E129G, W140C, W140G, W140R, W140X, E142X/K143X, G144E, Y145X, T147N, L155P, Y169D, Y170C, Y170P, A173D, Q174R, T1761, A183@, S184X, Q186X, L193@, L195P, R200X, E202X, R2031, A208D, C218X, Q220X, Q221X, H224Y, G226A, G227V, L231S, Q232X, A235P, S236X, V237E, T254M, G257D, G257S, and L258S, where X denotes a deletion of the amino acid and @ denotes insertions of one or more amino acids at these locations.

An additional aspect has one or more modifications (e.g. substitutions) selected from the group consisting of: E142M, E142V, E142C, E142I, G144Q, G144M, G144E, G144H, Y145P, Y145E, Y145N, T176G, R203Y, R203F, R203T, R203W, R203A, R203S, R203D, R207C, C218P, Q220P, Q220C, and Q220T.

An additional aspect has one or more modifications (e.g. substitutions) selected from the group consisting of: K143E, R203E, and R207E.

An additional aspect has one or more modifications (e.g. substitutions) selected from the group consisting of: M36R, K96N, A123E, H125R, V126D, V126A, S128R, E129G, W140C, W140G, W140R, W140X, E142X/K143X, G144E, Y145X T147N, L155P, Y169D, Y170C, Y170P, A173D, Q174R, T1761, A183@, S184X, Q186X, L193@, L195P, R200X, E202X, R2031, A208D, C218X, Q220X, Q221X, H224Y, G226A, G227V, L231S, Q232X, A235P, S236X, V237E, T254M, G257D, G257S, and L258S, where X denotes a deletion of the amino acid and @ denotes insertions of one or more amino acids at these positions.

An additional aspect has one or more modifications (e.g. substitutions) selected from the group consisting of: C218X, S128R, E129G, E142X/K143X, K143T, G144E, Y145X, Y145N, T147N, T176I, R203I, A208D, Q220X, and T254M.

An additional aspect has one or more modifications (e.g. substitutions) selected from the group consisting of: E142M, E142I, G144Q, G144H, Y145P, Y145N, T176G, R203T, R203D, R207C, Q220P, and Q220T.

A further aspect has one or more modifications (e.g. substitutions) selected from the group consisting of the following trimer interface residues: Q118, N119, Q121, A123, H125, Y145, T147, S149, Q166, G167, L168, Y170, Y172, Q174, T176, R181, P188, I190, L195, R203, I204, L205, L206, R207, A208, A209, N210, T211, S213, S214, A215, K216, P217, G219, Q220, Q221, S222, I223, H224, L225, G226, G227, V228, F229, E230, Q232, G250, T251, G252, F253, S255, L259, and L261.

An additional aspect has one or more modifications (e.g. substitutions) selected from the group consisting of the following trimer interface substitutions: Q118H, Q118E, Q118Q, Q118N, Q118A, Q118T, Q118S, Q118W, Q118F, Q118V, Q118G, Q118C, Q118K, Q118R, Q118Y, Q118D, Q118M, N119H, N119Q, N119L, N119N, N119R, N119D, N119A, N119S, N119M, N119K, N119G, N119E, Q121H, Q121Q, Q121T, Q121V, Q121Y, Q121M, Q121C, Q121D, Q121A, Q121E, Q1211, Q121N, Q121F, Q121L, Q121S, Q121G, A123A, H125H, H125N, H125M, Y145P, Y145E, Y145Y, Y145H, Y145N, T147V, T147T, T1471, S149S, S149A, Q166S, Q166G, Q166C, Q166D, Q166Q, Q166E, Q166A, Q166N, G167G, L168L, L168P, Y170Y, Y172Y, Q1741, Q174Q, Q174V, Q174L, Q174H, Q174K, T176A, T176S, T176G, T176T, R181G, R181S, R181C, R181A, S185R, S185S, S185N, S185K, S185H, S185T, S185A, S185Q, Q186H, Q186K, Q186R, Q186Q, Q186N, Q186M, Q186E, Q186T, Q186D, Q1861, Q186V, Q186L, Q186S, Q186A, Q186C, P188P, P188D, P188N, P188H, P188E, P188M, P188C, P188Q, P188K, P188T, P188A, P188V, P188S, I190V, I190I, I190Y, I190A, I190C, I190S, I190E, I190L, L195L, R200E, R200Q, R200H, R200D, R200N, R200M, R200S, R203Y, R203E, R203F, R203H, R203Q, R203T, R203W, R203A, R203N, R203S, R203R, R203C, R203D, I204E, I204V, I204P, I204L, I204M, I204I, I204T, I204Q, I204C, L205L, L206Q, L206L, L206M, L206E, R207R, R207C, R207N, A208A, A209A, A209S, N210Q, N210N, N210C, N210E, T211D, T211Y, T211E, S213S, S213D, S213A, S214S, S214P, S214D, S214N, S214A, S214E, S214Q, A215G, A215A, A215S, K216D, P217P, P217D, P217E, P217Y, P217Q, P217H, P217S, P217F, P217N, P217A, P217G, C218P, G219G, Q220P, Q220E, Q220D, Q220M, Q220Q, Q220N, Q220C, Q220T, Q221L, Q221Y, Q221M, Q221E, Q221S, Q221D, Q221F, S222S, S222A, S222T, S222C, I223I, H224F, H224H, L225L, G226G, G227G, V228V, F229F, E230P, E230H, E230M, E230E, E230Q, Q232D, Q232E, Q232H, Q232N, Q232Q, Q232Y, Q232V, Q232S, G250G, T251E, T251Q, T251M, T251D, T251P, T251V, T251N, T251T, T251H, T251I, T251A, T251L, T251S, T251R, T251G, G252G, F253F, F253E, F253Y, F253M, F253D, F253H, S255S, S255V, S255C, S255T, S255A, L259L, K260S, K260K, K260A, K260C, K260Q, K260M, K260N, K260E, L261V, L261L, L261I, L261T, L261C, L261A. Preferred additional variants are selected from the group consisting of: C194A, C194Q, C194K, and C194M.

In a further aspect, the invention provides double variants (as well as higher, as outlined herein) selected from the group consisting of: C178S/C218E, C178S/C218Q, C178T/C218E, C178T/C218Q, C178A/C218E, and C178A/C218Q.

In a further aspect, the invention provides compositions with the formula: Fx(45-114)-Vb(115)-Vb (116)-Vb (117)-Vb(118)-Vb(119)-Vb(120)-Vb(121)-Fx(122)-Vb(123)-Fx(124)-Vb(125)-Fx(126)-Vb(127)-Vb(128)-Vb(129)-Vb(130)-Fx(131)-Vb(132)-Vb(133)-Vb(134)-Fx(135-136)-Vb(137)-Fx(138-139)-Vb(140)-Vb(141)-Vb(142)-Vb(143)-Vb(144)-Vb(145)-Vb(146)-Vb(147)-Vb(148)-Vb(149)-Fx(150)-Vb(151)-Vb(152)-Fx(153-154)-Vb(155)-Fx(156)-Vb(157)-Fx(158-165)-Vb(166)-Vb(167)-Vb(168)-Vb(169)-Vb(170)-Fx(171)-Vb(172)-Vb(173)-Vb(174)-Fx(175)-Vb(176)-Vb(177)-Vb(178)-Vb(179)-Fx(180)-Vb(181)-Vb(182)-Vb(183)-Vb(184)-Vb(185)-Vb(186)-Vb(187)-Vb(188)-Vb(189)-Vb(190)-Vb(191)-Vb(192)-Vb(193)-Vb(194)-Vb(195)-Vb(196)-Vb(197)-Vb(198)-Vb(199)-Vb(200)-Vb(201)-Vb(202)-Vb(203)-Vb(204)-Vb(205)-Vb(206)-Vb(207)-Vb(208)-Vb(209)-Vb(210)-Vb(211)-Fx(212)-Vb(213)-Vb(214)-Vb(215)-Vb(216)-Vb(217)-Vb(218)-Vb(219)-Vb(220)-Vb(221)-Vb(222)-Vb(223)-Vb(224)-Vb(225)-Vb(226)-Vb(227)-Vb(228)-Vb(229)-Vb(230)-Vb(231)-Vb(232)-Vb(233)-Fx(234-241)-Vb(242)-Vb(243)-Vb(244)-Vb(245)-Vb(246)-Vb(247)-Vb(248)-Vb(249)-Vb(250)-Vb(251)-Vb(252)-Vb(253)-Vb(254)-Vb(255)-Fx(256-258)-Vb(259)-Vb(260)-Vb(261)

wherein

Fx(45-115) is optionally present, and if present, comprises the human amino acid sequence of CD40L at positions 45-115;
Vb(116) is selected from the group consisting of G and C;
Vb(117) is selected from the group consisting of D and C;
Vb(118) is selected from the group consisting of Q, C, H, E, N, A, T, S, W, F, V, G, K, R, Y, D, and M;
Vb(119) is selected from the group consisting of N, H, Q, L, N, R, D, A, S, M, K, G, and E;
Vb(120) is selected from the group consisting of P, C, R, Q, N, E, D, K, G, and S;
Vb(121) is selected from the group consisting of Q, H, T, V, Y, M, C, D, A, E, I, N, F, L, S, and G;
Fx(122) comprises the human amino acid sequence of CD40L at position 122;
Vb(123) is selected from the group consisting of A and E;
Fx(124) comprises the human amino acid sequence of CD40L at position 124;
Vb(125) is selected from the group consisting of H, R, N, and M;
Fx(126) is selected from the group consisting of V, D, and A;
Vb(127) is selected from the group consisting of H, Q, E, C, S, D, and I;
Vb(128) is selected from the group consisting of S and R;
Vb(129) is selected from the group consisting of E and G;
Vb(130) is selected from the group consisting of P, Q, K, M, N, C, R, E, D, and A;
Fx(131) comprises the human amino acid sequence of CD40L at position 131;
Vb(132) is selected from the group consisting of C and S;
Vb(133) is selected from the group consisting of C and K;
Vb(134) is selected from the group consisting of C, Q, E, D, R, N, K, S, G, and T;
Fx(135-136) comprises the human amino acid sequence of CD40L at positions 135-136;
Vb(137) is selected from the group consisting of R, K, S, and V;
Fx(138-139) comprises the human amino acid sequence of CD40L at positions 138-139;
Vb(140) is selected from the group consisting of W, C, G, R, and a deletion;
Vb(141) comprises any amino acid;
Vb(142) is selected from the group consisting of M, V, C, I, E, and a deletion;
Vb(143) is selected from the group consisting of C, E, K, and a deletion;
Vb(144) is selected from the group consisting of Q, M, E, H, K, R, and G;
Vb(145) is selected from the group consisting of P, E, N, C, D, H, Y, and a deletion;
Vb(146) is selected from the group consisting of S, C, and N;
Vb(147) is selected from the group consisting of T, V, I, and N;
Vb(148) is selected from the group consisting of M and K;
Vb(149) is selected from the group consisting of S and A;
Fx(150) comprises the human amino acid sequence of CD40L at position 150;
Vb(151) comprises any amino acid;
Vb(152) is selected from the group consisting of L, E, and Q;
Fx(153-154) comprises the human amino acid sequence of CD40L at positions 153-154;
Vb(155) is selected from the group consisting of L and P;
Fx(156) comprises the human amino acid sequence of CD40L at position 156;
Vb(157) comprises any amino acid;
Fx(158-165) comprises the human amino acid sequence of CD40L at positions 158-165;
Vb(166) is selected from the group consisting of Q, S, G, C, D, Q, E, A, and N;
Vb(167) comprises any amino acid;
Vb(168) is selected from the group consisting of C, P, and L;
Vb(169) is selected from the group consisting of Y and D;
Vb(170) is selected from the group consisting of Y, C, and P;
Fx(171) comprises the human amino acid sequence of CD40L at position 171;
Vb(172) comprises any amino acid;
Vb(173) is selected from the group consisting of A and D;
Vb(174) is selected from the group consisting of Q, R, I, V, L, H, and K;
Fx(175) comprises the human amino acid sequence of CD40L at position 175;
Vb(176) is selected from the group consisting of T, G, I, A, S, and G;
Vb(177) comprises any amino acid;
Vb(178) is selected from the group consisting of C, G, S, D, E, H, Q, N, K, Y, M, and R;
Vb(179) comprises any amino acid;
Fx(180) comprises the human amino acid sequence of CD40L at position 180;
Vb(181) is selected from the group consisting of R, G, S, C, and A;
Vb(182) is selected from the group consisting of E and C;
Vb(183) is selected from the group consisting of A, Q, E, R, D, N, K, S, G, and an insertion;
Vb(184) is selected from the group consisting of S, C, L, R, M, and deletion;
Vb(185) is selected from the group consisting of S, R, H, N, K, H, T, A, and Q;
Vb(186) is selected from the group consisting of Q, T, I, V, L, A, C, H, K, R, N, M, E, D, S, and deletion;
Vb(187) is selected from the group consisting of A, E, H, W, Q, M, D, L, R, N, and K;
Vb(188) is selected from the group consisting of P, D, N, H, E, M, C, Q, K, T, A, V, S, and G;
Vb(189) comprises any amino acid;
Vb(190) is selected from the group consisting of I, Y, A, C, S, E, and V;
Vb(191) comprises any amino acid;
Vb(192) comprises any amino acid;
Vb(193) is selected from the group consisting of L and an insertion;
Vb(194) is selected from the group consisting of C, A, Q, K, and M;
Vb(195) is selected from the group consisting of L and P;
Vb(196) comprises any amino acid;
Vb(197) comprises any amino acid;
Vb(198) is selected from the group consisting of C, E, D, N, Q, and P;
Vb(199) comprises any amino acid;
Vb(200) is selected from the group consisting of R, D, M, S, Q, H, N, and a deletion;
Vb(201) is selected from the group consisting of F, R, H, E, N, Q, D, K, S, T, and G;
Vb(202) is selected from the group consisting of E and a deletion;
Vb(203) is selected from the group consisting of R, Y, F, T, W, A, S, D, C, I, E, N, H, and Q;
Vb(204) is selected from the group consisting of I, E, Q, S, V, P, L, M, T, Q, and C;
Vb(205) comprises any amino acid;
Vb(206) is selected from the group consisting of L, Q, M, and E;
Vb(207) is selected from the group consisting of R, C, E, and N;
Vb(208) is selected from the group consisting of A and D;
Vb(209) is selected from the group consisting of C, S, and A;
Vb(210) is selected from the group consisting of N, Q, C, and E;
Vb(211) is selected from the group consisting of C, D, Y, and E;
Fx(212) comprises the human amino acid sequence of CD40L at position 212;
Vb(213) is selected from the group consisting of S, D, and A;
Vb(214) is selected from the group consisting of S, P, R, D, N, A, E, and Q;
Vb(215) is selected from the group consisting of C, G, A, and S;
Vb(216) is selected from the group consisting of K and D;
Vb(217) is selected from the group consisting of P, D, E, Q, S, N, G, Y, H, F, and A;
Vb(218) is selected from the group consisting of C, P, D, G, and a deletion;
Vb(219) comprises any amino acid;
Vb(220) is selected from the group consisting of Q, P, C, T, E, D, M, N, and a deletion;
Vb(221) is selected from the group consisting of Q, L, Y, M, E, S, D, F, and a deletion;
Vb(222) is selected from the group consisting of S, A, T, and C;
Vb(223) is selected from the group consisting of P, E, D, S, Q, N, R, and G;
Vb(224) is selected from the group consisting of H, Y, and F;
Vb(225) comprises any amino acid;
Vb(226) is selected from the group consisting of G and A;
Vb(227) is selected from the group consisting of G and V;
Vb(228) comprises any amino acid;
Vb(229) comprises any amino acid;
Vb(230) is selected from the group consisting of E, P, H, M, and Q;
Vb(231) is selected from the group consisting of L and S;
Vb(232) is selected from the group consisting of Q, D, E, H, N, Y, V, S, and a deletion;
Vb(233) comprises any amino acid;
Fx(234-241) comprises the human amino acid sequence of CD40L at positions 234-241;
Vb(242) comprises any amino acid;
Vb(243) comprises any amino acid;
Vb(244) comprises any amino acid;
Vb(245) is selected from the group consisting of S, R, P, H, M, V, L, Y, and I;
Vb(246) comprises any amino acid;
Vb(247) comprises any amino acid;
Vb(248) is selected from the group consisting of S and C;
Vb(249) is selected from the group consisting of H and D;
Vb(250) comprises any amino acid;
Vb(251) is selected from the group consisting of T, E, Q, M, D, P, H, I, L, R, V, N, A, S, and G;
Vb(252) comprises any amino acid;
Vb(253) is selected from the group consisting of F, E, D, H, Y, and M;
Vb(254) is selected from the group consisting of T and M;
Vb(255) is selected from the group consisting of S, U, C, T, and A;
Fx(256-258) comprises the human amino acid sequence of CD40L at positions 256-258;
Vb(259) comprises any amino acid;
Vb(260) is selected from the group consisting of K, S, A, C, Q, M, N, and E;
Vb(261) is selected from the group consisting of L, N, S, D, V, I, T, C, and A.

In the above formula, another preferred aspect is the presence of the wild-type residue at any position reciting “any amino acid”, e.g. Vb positions 141, 151, 157, 167, 172, 177, 179, 189, 191, 192, 196, 197, 199, 205, 219, 225, 228, 229, 233, 242, 243, 244, 246, 247, 250, 252 and 259 can be any amino acid, and in an alternative preferred embodiment are human wild-type amino acids.

An additional aspect has one or more polymeric moieties attached at one or more sites selected from the group comprising Gly116, Asp117, Gln118, Pro120, Gln121, Ala130, Ser132, Lys133, Thr134, Lys143, Tyr145, Asn151, Asn157, Leu168, Glu182, Ala183, Ser185, Gln186, Ala187, Pro198, Gly199, Phe201, Arg203, Ala209, Thr211, Ser213, Ala215, Pro217, Cyd218, Gln220, His224, Val228, Glu230, Pro233, Ser245, Thr251, Gly252, and Leu261. The amino acid at the cited position may be wild-type, naturally occurring (e.g. a substituted cysteine) or a non-natural amino acid such as p-acetyl-L-phenylalanine.

In accordance with the objects outlined above, the present invention provides CD40L variant proteins comprising amino acid sequences with at least one amino acid change compared to the wild-type CD40L proteins.

### BRIEF DESCRIPTION OF THE DRAWINGS

FIG. 1a shows the full-length amino acid sequence of wild-type human CD40L (amino acids 1-261 of GenBank accession 153476; SEQ ID NO: 1). FIG. 1b shows the amino acid sequence of wild-type human CD40L ECD (amino acids 45-261 of Genbank accession 153476; SEQ ID NO: 2).

FIGS. 2A-E shows a Multiple Sequence Alignment (MSA) of human TNFSF members. Amino acids in the alignment are differentially highlighted according to 6 groupings of physicochemical properties as follows: nonpolar (A, C, I, L, M, V), aromatic (F, W, Y), neutral polar (N, Q, S, T), charged positive (H, K, R), charged Negative (D, E), and conformational (G, P). FIG. 2 also shows position numberings of each individual sequence. For TNF-α (TNFA) and TNFB (LT-α), the numbering is based on current convention. For all other sequences, the numbering is based on the full-length precursor sequence of the protein. For sequences in which a structure of the ligand-receptor complex has been determined experimentally (e.g. TNFB, TRAIL, BLyS), or is readily modeled (e.g. TNFA), position numbers that lie at a ligand-receptor interface are highlighted in gray. Positions highlighted for RANKL have been experimentally determined to affect receptor binding. Receptor interfaces, highlighted in black, are used to define general receptor contact regions of the TNF superfamily ligands. A generic numbering system, beginning with position number 1, is also included above the MSA for reference.

FIG. 3 shows PEGylation calculations to identify sites for attachment of PEG, or other polymers. Cysteine substitutions are considered at each position in CD40L with 2,000 M.W. PEG chains linked via maleimide. PEG chains are added to the protein in random conformations. Shown in the top panel is the percent of PEG chains that do not clash with a monomeric CD40L protein, when the PEG chains are attached at each position. Shown in the bottom panel is the percent of PEG chains that do not clash with the trimeric CD40L protein. Preferable positions for PEGylation are identify by high percent non-clashing values in the bottom panel.

FIG. 4 shows energies of substitutions of CD40L positions with various amino acids. The energies were calculated with PDA® algorithms and are shown below the amino acid substituted at each position in CD40L. Wild-type, human, CD40L amino acids and positions are shown in the first two columns. Favorable substitutions have lower energies and are found toward the left side of the table. The most favored amino acid at each position has an energy of 0.0 kcal/mole.

FIG. 5 depicts Table 1, referenced in the specification.

FIG. 6 depicts a table of glycosylation sites and preferred substitutions.

FIG. 7 depicts a table of preferred variant positions (using CD40L numbering from FIG. 3, with corresponding TNFA numbering). “Vb” stands for “variable”, e.g. suitable for modification as outlined herein, and “fx” stands for fixed, e.g. the wild-type sequence at these positions. The extracellular domain spans from residue 45-261, but residues 45-115 (116, 117, 118, 119 or 120) can be optional. Each set of preferred amino acids includes the wild-type residue, such that additional “fixed” sequences can be made; for example, if the variant is the double variant C178S/C218E, then the formula comprises Fx(45-177)-Vb(178)-Fx(179-217)-Vb(218)-Fx(219-261), with Vb(178) being S and Vb(218) being E. In addition, Vb positions which carry only a single amino acid option or are blank can be any amino acid. Additionally, these positions (Vb positions 141, 151, 157, 167, 172, 177, 179, 189, 191, 192, 196, 197, 199, 205, 219, 225, 228, 229, 233, 242, 243, 244, 246, 247, 250, 252 and 259) are in some embodiments human wild-type amino acids (e.g. Vb141 is the wild-type, A).

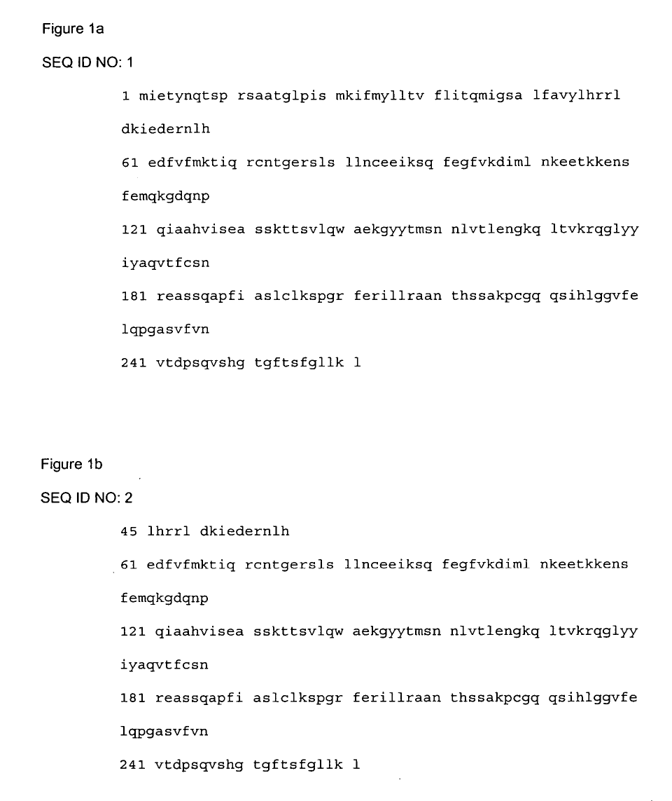

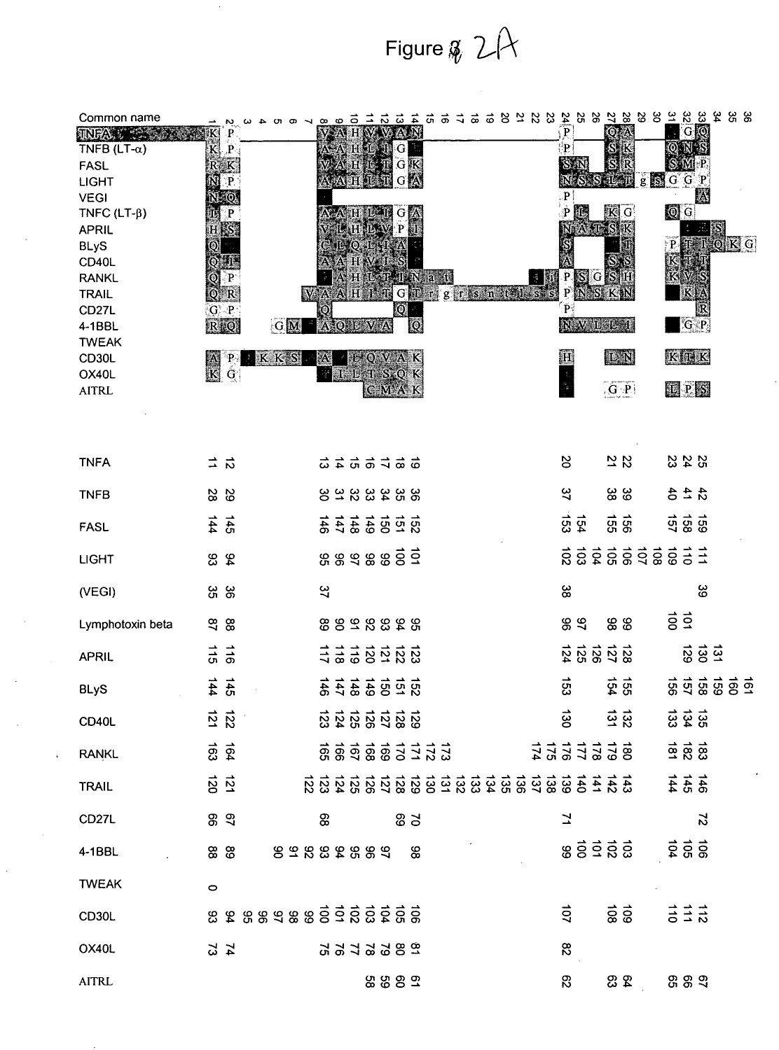

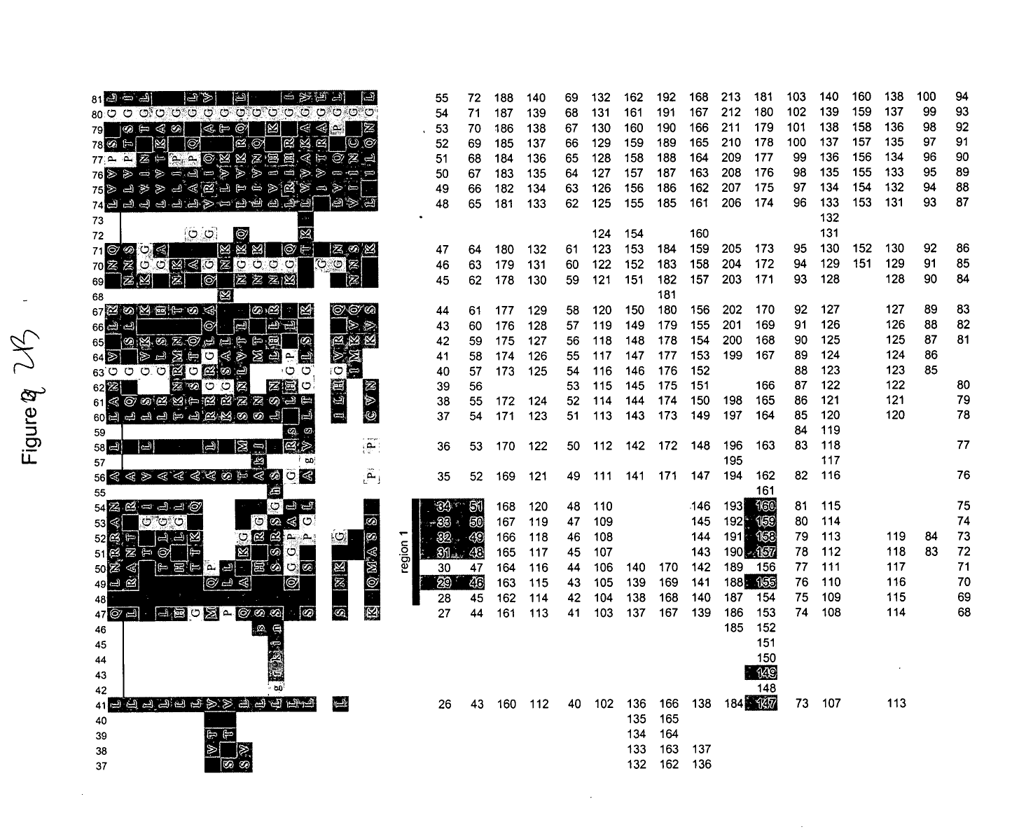

### DETAILED DESCRIPTION OF THE INVENTION

By “CD40” herein is meant a cell-surface receptor activator of NF-κB. The CD40 protein (or nucleic acid) may be from any number of organisms, with CD40 proteins from mammals being particularly preferred. Suitable mammals include, but are not limited to, rodents (rats, mice, hamsters, guinea pigs, etc.), primates, farm animals (including sheep, goats, pigs, cows, horses, etc); and in the most preferred embodiment, from humans (the sequence of which is depicted in the figures). As will be appreciated by those in the art, CD40 proteins based on CD40 proteins from mammals other than humans may find use in animal models of human disease.

By “CD40L” herein is meant a ligand for CD40. Again, CD40L sequences from humans are preferred (sequences depicted in the Figures), but CD40L sequences from organisms outlined above are also included. As is more fully outlined below, CD40L proteins trimerize (although higher oligomers may also occur) to activate the CD40L receptors. Thus, CD40L proteins occur as monomers as well as oligomers, generally trimers. As more fully outlined below, variant CD40L protein monomers can be made which physically interact with other monomers (including other variant CD40L monomers, wild-type monomers, or wild-type monomers from different species) to form mixed oligomers or trimers.

By “variant CD40L monomer proteins” or grammatical equivalents herein is meant a CD40L monomer protein that contains at least one modification, including substitutions, insertions and deletions. Thus, variant CD40L proteins means non-naturally occurring CD40L proteins that differ from the wild-type CD40L protein by at least 1 modification, including but not limited to amino acid substitution, insertion, or deletion. CD40L variants are characterized by the predetermined nature of the variation, a feature that sets them apart from naturally occurring allelic or interspecies variation of the CD40L protein sequence. The CD40L variants typically either exhibit the same qualitative biological activity as the naturally occurring CD40L or have been specifically engineered to have alternate biological properties. Variant CD40L proteins are derived from the extracellular domain of wild-type CD40L SEQ ID NO: 2 (amino acids 45-261). In preferred embodiments, variant CD40L proteins include the TNF homology domain (THD) composed of amino acids 123-161. However, the variant CD40L proteins may contain insertions or deletions at the N-terminus, C-terminus, or internally. Thus, in a preferred embodiment, variant CD40L proteins have at least 1 residue that differs from the human CD40L sequence, with at least 2, 3, 4, or 5 different residues being more preferred. Variant CD40L proteins may comprise one domain or multiple domains connected by linker sequences. Variant CD40L proteins may contain further modifications, for instance modifications that alter stability or immunogenicity or which enable or disrupt posttranslational modifications such as PEGylation or glycosylation.

In general, deletions include portions of CD40L that are not extracellular, and include substitutions in residues corresponding to monomer association sites and receptor recognition and/or binding sites. Preferred variants are further described below, and include both conservative and non-conservative substitutions, with the latter being preferred.

By “extracellular domain” or “ECD” as used herein is meant the segment of protein existing predominantly outside the cell. For transmembrane proteins, this segment can be tethered to the cell through a transmembrane domain or released from the cell through proteolytic digestion. Alternatively, the extracellular domain could comprise the whole protein or amino acid segments thereof when secreted from the cell. In general, CD40L members are expressed as type II transmembrane proteins (extracellular C terminus). Soluble forms of CD40L proteins may result from proteolytic cleavage of the signal propeptide by matrix metalloproteinases or directly by recombinant methods. Unless otherwise disclosed, the variant CD40L proteins of the present invention are soluble forms or functional equivalents thereof. That is, the variants of the present invention do not comprise transmembrane domains unless specifically noted.

By “agonists of wild-type CD40L” or grammatical equivalents thereof, herein is meant variants of CD40L which themselves or in combination with wild-type CD40L appreciably activate CD40L receptors. In a preferred embodiment, activation of the CD40L receptors by agonistic CD40L variants is between 50% and 110% of the activation that would be attained with an equal amount of wild-type CD40L protein, with between 80% and 100% being especially preferred.

By “superagonists of wild-type CD40L” or grammatical equivalents thereof, herein is meant variants of CD40L that enhance the activation of CD40L receptors signaling by wild-type CD40L proteins or that themselves activate the CD40L receptor more strongly than an equal amount of wild-type CD40L. In a preferred embodiment the CD40L superagonist proteins yield at least 10% more activity than wild-type CD40L, with at least 50%, 100% or 200% increases in activity being especially preferred.

By “altered property” or grammatical equivalents thereof herein is meant, in the context of a protein, any characteristic or attribute of a protein that differs from the corresponding property of a naturally occurring protein. These properties include, but are not limited to cytotoxic activity, oxidative stability, substrate specificity, substrate binding or catalytic activity, thermal stability, alkaline stability, pH activity profile, resistance to proteolytic degradation, kinetic association (kₒₙ) and dissociation (K_{off}) rates, immunogenicity, binding affinity and selectivity, ability to be secreted, ability to be displayed on the surface of a cell, ability to oligomerize, ability to signal, ability to stimulate cell proliferation, ability to activate receptors, ability to inhibit cell proliferation, ability to induce apoptosis, ability to be modified by phosphorylation or glycosylation, and the ability to treat disease. Unless otherwise specified, a property of a CD40L variant is considered to be “altered” when the property exhibits preferably at least a 5%, more preferably 50%, and most preferably at least a 2-fold increase or decrease relative to the corresponding property in the wild-type CD40L protein. For example, a change in binding affinity is evidenced by at least a 5% or greater increase or decrease in binding affinity to wild-type CD40 and/or alpha IIb-beta3 integrin receptors.

By “antagonists of wild-type CD40L” or grammatical equivalents thereof herein is meant variants of CD40L that inhibit or significantly decrease the activation of CD40L receptors signaling by wild-type CD40L proteins. Both dominant negative CD40L variants and competitive inhibitors of CD40L are antagonists of wild-type CD40L. Furthermore, antagonists of wild-type CD40L do not appreciably activate the CD40L receptors and initiate the CD40L signaling pathway(s). In a preferred embodiment, at least a 50% decrease in receptor activation relative to wild-type CD40L is seen, with greater than 50%, 76%, 80-90% being preferred.

By “competitive inhibitor CD40L variants” or “ciCD40L” or grammatical equivalents thereof herein is meant variants that compete with naturally occurring CD40L protein for binding to the CD40L receptors without activating CD40 signaling, thereby limiting the ability of naturally occurring CD40L to bind and activate the CD40L receptors. In general, ciCD40L proteins are included within the definition of variant CD40L proteins.

By “conformer” herein is meant a protein that has a protein backbone three-dimensional structure that is virtually the same as a reference protein but that has significant differences in the amino acid sequence.

By “control sequences” herein is meant nucleic acid sequences necessary for the expression of an operably linked coding sequence in a particular host organism. Control sequences include, but are not limited to, promoters, enhancers, and ribosome-binding sites.

By “epitope” herein is meant a portion of a protein that mediates an immune response. An epitope may serve as a binding site for an antibody, T-cell receptor, and/or MHC molecule.

By “exposed residues” as used herein is meant those residues whose side chains are significantly exposed to solvent. In a preferred embodiment, at least 30 Å² of solvent exposed area is present, with greater than 50 Å² or 75 Å² being especially preferred. In an alternate embodiment, at least 50% of the surface area of the side chain is exposed to solvent, with greater than 75% or 90% being preferred.

By “gene therapy” herein is meant the one time or repeated administration of a therapeutically effective DNA, mRNA, or other nucleic acid. In one embodiment, genes are introduced into cells in order to achieve in vivo synthesis of a therapeutically effective genetic product, for example for replacement of a defective gene. Antisense RNA and DNA can be used as therapeutic agents for blocking the expression of certain genes in vivo. In alternate embodiments, double stranded RNA derived from a target gene can block the expression of a target gene in vivo, and ribozymes can be used to process or degrade a target gene of interest. Antisense nucleic acids can be designed to structural genes or regulatory regions thereof.

By “hydrophobic residues” or “nonpolar residues” as used herein is meant valine, leucine, isoleucine, methionine, phenylalanine, tyrosine, and tryptophan.

By “increase in polar character” as used herein is meant any of the following: (1) replacement of hydrophobic residues with neutral polar or charged residues or (2) the replacement of neutral polar residues with charged residues.

By “labeled” herein is meant that a protein has at least one element, isotope or chemical compound attached to enable the detection and/or purification of the protein. In general, labels include, but are not limited to, a) isotopic labels, which may be radioactive or heavy isotopes; b) immune labels, which may be antibodies or antigens; c) colored or fluorescent dyes, d) enzymes, e) particles such as colloids, magnetic particles, etc.

By “linker”, “linker sequence”, “spacer”, “tethering sequence” or grammatical equivalents thereof, herein is meant a molecule or group of molecules (such as a monomer or polymer) that connects two molecules and often serves to place the two molecules in a preferred configuration. In one aspect of this embodiment, the linker is a peptide bond. Choosing a suitable linker for a specific case where two polypeptide chains are to be connected depends on various parameters, e.g., the nature of the two polypeptide chains (e.g., whether they naturally oligomerize (e.g., form a dimer or not), the distance between the N- and the C-termini to be connected if known from three-dimensional structure determination, and/or the stability of the linker towards proteolysis and oxidation.

Furthermore, the linker may contain amino acid residues that provide flexibility. Thus, the linker peptide may predominantly include the following amino acid residues: Gly, Ser, Ala, or Thr. These linked CD40L proteins have constrained hydrodynamic properties, that is, they form constitutive dimers, and thus efficiently interact with other naturally occurring CD40L proteins to form a dominant negative heterotrimer.

By “mixed trimers” (frequently used interchangeably with mixed oligomers herein) is meant trimers that are composed of one or two monomers of wild-type CD40L and one or two monomers of variant CD40L protein.

By “nonconservative modification” herein is meant a modification in which the wild-type residue and the mutant residue differ significantly in one or more physical properties, including hydrophobicity, charge, size, and shape. For example, modifications from a polar residue to a nonpolar residue or vice-versa, modifications from positively charged residues to negatively charged residues or vice versa, and modifications from large residues to small residues or vice versa are nonconservative modifications.

Conservative modifications are generally those shown below, however, as is known in the art, other substitutions may be considered conservative:

| | |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Modifications of the proteins are preferably substitutions and may include those to surface, boundary and core areas of a CD40L member. See, for example, U.S. Pat. Nos. 6,188,965; 6,269,312, and 6,403,312, hereby incorporated by reference. In another preferred embodiment, modifications may be made to surface residues, particularly when alterations to binding properties are desired (either to other monomers or to the receptor).

By “nucleic acid” herein is meant DNA, RNA, and related molecules, which contain deoxy- and/or ribonucleotides. In some cases, for example for use with antisense nucleic acids, nucleic acid analogs may be used.

By “operably linked” herein is meant that a nucleic acid is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation.

By “patient” herein is meant both humans and other animals, particularly mammals as outlined herein, and non-animal organisms, with humans being preferred.

By “pharmaceutically acceptable salt” as used herein refers to those salts that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like, or derived from inorganic bases such as sodium, potassium, lithium, ammonium, calcium, magnesium, iron, zinc, copper, manganese, aluminum salts and the like, as well as salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

By “polar residues” herein is meant serine, threonine, histidine, aspartic acid, asparagine, glutamic acid, glutamine, arginine, and lysine.

As used in this invention, the term “polymer” and “polymeric moiety” or its grammatical equivalents means any non-monomeric moiety that is attachable to a protein, is at least partially soluble and has the appropriate flexibility to achieve a desired function. The polymer can be homopolymeric or heteropolymeric. In a preferred embodiment of the invention, polymer moieties may include but are not limited to alcohol such as glycols moieties and carbohydrate moieties. A preferred range of molecular weight is about 1000 Daltons to about 100,000 Daltons. The polymer may be unbranched, branched, or labile, including both internal lability, e.g. cleavage upon introduction into a patient, as well as attachment lability, wherein the linkage between the protein and the polymer is reversible The polymer may have organic or inorganic components or moieties. In a preferred embodiment, the polymer is pharmaceutically acceptable and may be attached to therapeutic proteins. A preferred example of a suitable polymer is polyethylene glycol (PEG) and its derivatives. For ease of discussion, the term “PEG” will be used, but is meant to include the scope of the term “polymer” as defined above. Examples of suitable polymers include but are not limited to, example Roberts, M. J. et al. (2002) “Chemistry for peptide and protein PEGylation” Adv. Drug Deliv. Rev. 54, 459-476 and Kinstler, O. et al. (2002) “Mono-N-terminal poly(ethylene glycol)-protein conjugates” Adv. Drug Deliv. Rev. 54; U.S. Ser. No. 60/360,722; U.S. Pat. No. 5,795,569; U.S. Pat. No. 5,766,581; EP 01064951; U.S. Pat. No. 6,340,742; WO 00176640; WO 002017; EP0822199A2; WO 0249673A2; U.S. Pat. No. 4,002,531; U.S. Pat. No. 5,183,550; U.S. Pat. No. 5,985,263; U.S. Pat. No. 5,990,237; U.S. Pat. No. 6,461,802; U.S. Pat. No. 6,495,659; U.S. Pat. No. 6,448,369; U.S. Pat. No. 6,437,025; U.S. Pat. No. 5,900,461; U.S. Pat. No. 6,413,507; U.S. Pat. No. 5,446,090; U.S. Pat. No. 5,672,662; U.S. Pat. No. 6,214,966; U.S. Pat. No. 6,258,351; U.S. Pat. No. 5,932,462; U.S. Pat. No. 5,919,455; U.S. Pat. No. 6,113,906; U.S. Pat. No. 5,985,236; WO 9428024A1; U.S. Pat. No. 6,340,742; U.S. Pat. No. 6,420,339; and WO 0187925A2, all hereby incorporated by reference. PEG derivatives can include heteroatoms and substitution groups for hydrogen atoms.

By “protein” herein is meant at least two covalently attached amino acids, which includes proteins, polypeptides, oligopeptides and peptides. The protein may be made up of naturally occurring amino acids and peptide bonds, or synthetic peptidomimetic structures, i.e., “analogs” such as peptoids [see Simon et al., Proc. Natl. Acd. Sci. U.S.A. 89(20:9367-71 (1992)], generally depending on the method of synthesis. For example, homo-phenylalanine, citrulline, and noreleucine are considered amino acids for the purposes of the invention. “Amino acid” also includes amino acid residues such as proline and hydroxyproline. Both D- and L-amino acids may be utilized.

By “CD40L non-agonists” or grammatical equivalents thereof herein is meant variants of CD40L that do not appreciably activate CD40L receptors. In a preferred embodiment, activation of the CD40L receptors by non-agonistic CD40L variants is at most 50% of the activation that would be attained with an equal amount of wild-type CD40L protein, with at least less than 20% or 10% being especially preferred.

By “CD40L related disorder” or “CD40L responsive disorder” or grammatical equivalents thereof herein is meant a disorder that may be ameliorated, prevented, or treated by the administration of a pharmaceutical composition comprising a variant CD40L protein. CD40L-related diseases and disorders include but are not limited to cardiovascular conditions.

By “reduction in hydrophobicity” as used herein is meant the removal of hydrophobic chemical groups, the addition of polar or charged chemical groups, or the replacement of hydrophobic residues with more polar or charged residues.

By “soluble expression” or grammatical equivalents thereof as used herein is meant that the CD40L variant protein is expressed in soluble form (as opposed to forming aggregates or inclusion bodies). In a preferred embodiment, greater than 5% of the expressed CD40L variant protein is soluble, with at least 50%, 75% or 90% being especially preferred. In an alternate embodiment, the total yield of soluble protein is at least 0.01 mg/L of culture, or preferably at least 0.1 or 1.0 mg/L of culture. In a preferred embodiment, CD40L proteins that express solubly can be released, in soluble form, from cells using any non-denaturing buffer (that is, buffers that contain less than 1 M guanidinium or urea).

By “treatment” herein is meant to include therapeutic treatment, as well as prophylactic, or suppressive measures for the disease or disorder. Thus, for example, successful administration of a variant CD40L protein prior to onset of the disease may result in treatment of the disease. As another example, successful administration of a variant CD40L protein after clinical manifestation of the disease to combat the symptoms of the disease comprises “treatment” of the disease. “Treatment” also encompasses administration of a variant CD40L protein after the appearance of the disease in order to eradicate the disease. Successful administration of an agent after onset and after clinical symptoms have developed, with possible abatement of clinical symptoms and perhaps amelioration of the disease, further comprises “treatment” of the disease.

By “wild-type” herein is meant an amino acid sequence or a nucleotide sequence that is found in nature and includes allelic variations; that is, an amino acid sequence or a nucleotide sequence that has not been intentionally modified. In a preferred embodiment, the wild-type sequence is the most prevalent human sequence.

The present invention is directed to variant CD40L monomers and mixed oligomers containing the variant CD40L monomers that modify receptor activation. In preferred embodiments, the mixed oligomers have either antagonist or agonist activity. The therapeutic strategy is based on the design of novel CD40L variants that have altered receptor binding and/or activation properties as compared to naturally occurring CD40L proteins, and the ability to oligomerize with naturally occurring CD40L proteins. In other words, CD40L variants that result in reduced activation of (preferably do not substantially activate) CD40L receptors (as compared to a naturally occurring CD40L protein) will exchange with at least one naturally occurring CD40L protein and sequester it into reduced or inactive hetero-oligomers, inhibiting the oligomer's biological activity, e.g. the ability to bind and/or activate the receptor. Similarly, agonists can be done in a similar manner.

The CD40L variants of the present invention may be designed by modifying CD40L proteins at key receptor contact points in order to alter (disrupt or enhance, depending on the desired outcome) their ability to bind or activate the receptor. The exchange and physical interaction of these oligomeric CD40L variants with naturally occurring CD40L proteins results in altered activity of the naturally occurring CD40L oligomeric proteins. To help accomplish this goal more effectively, the CD40L variants can be designed to preferentially hetero-oligomerize with naturally occurring CD40L proteins. Alternatively, the variants may be designed to bind each other and “swamp” out the effect of any naturally occurring CD40L proteins due to the amount of variant oligomers present; e.g. equilibria favors the binding of the variant mixed oligomers.

Accordingly, the present invention provides methods and compositions utilizing variants of an extracellular domain of a CD40L protein. Especially preferred variants include antagonists (or non-agonists), and more specifically competitive antagonists. In addition, other preferred variants substantially reduce binding to the integrin alpha IIb-beta3 integrin. Other embodiments include variants that bind to CD40.

One aspect of the present invention is the generation of variants of human CD40L that express as soluble components from host systems, preferably bacteria and mammalian host cells such as CHO cells. A further aspect of the present invention is the generation of variants of human CD40L that act as antagonists of the wild-type CD40L and/or CD40 or variants of human CD40L that act as superagonists of CD40 signaling.

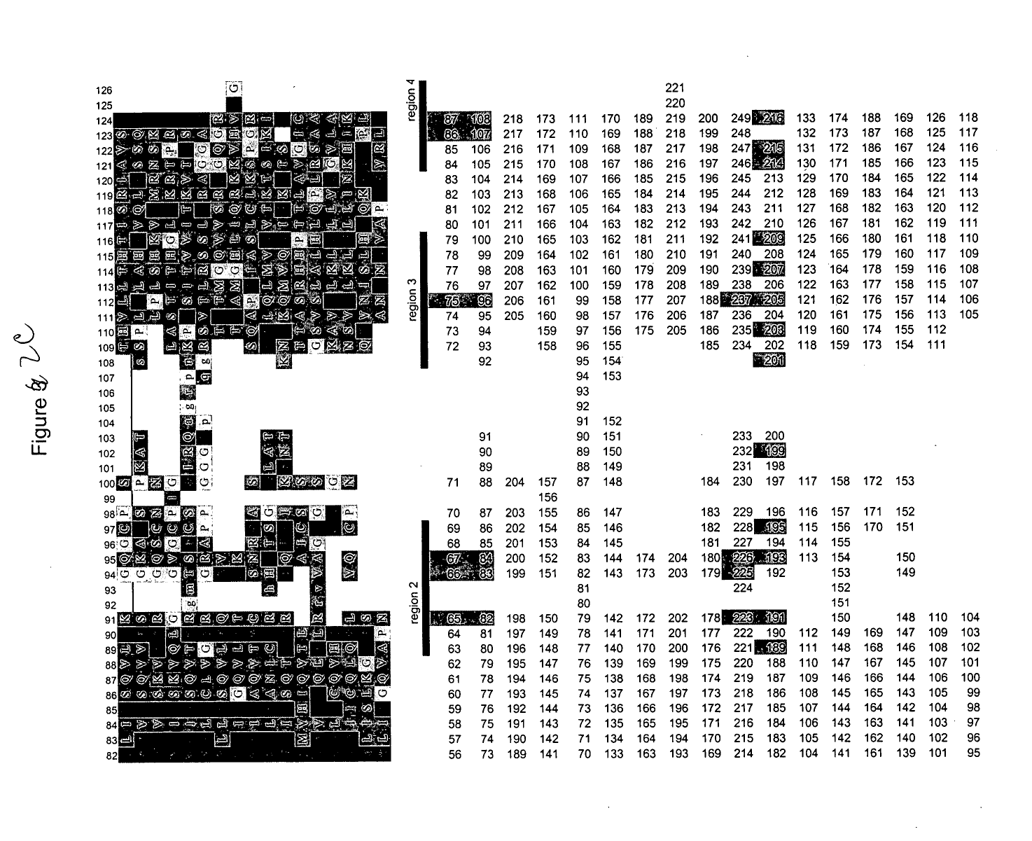

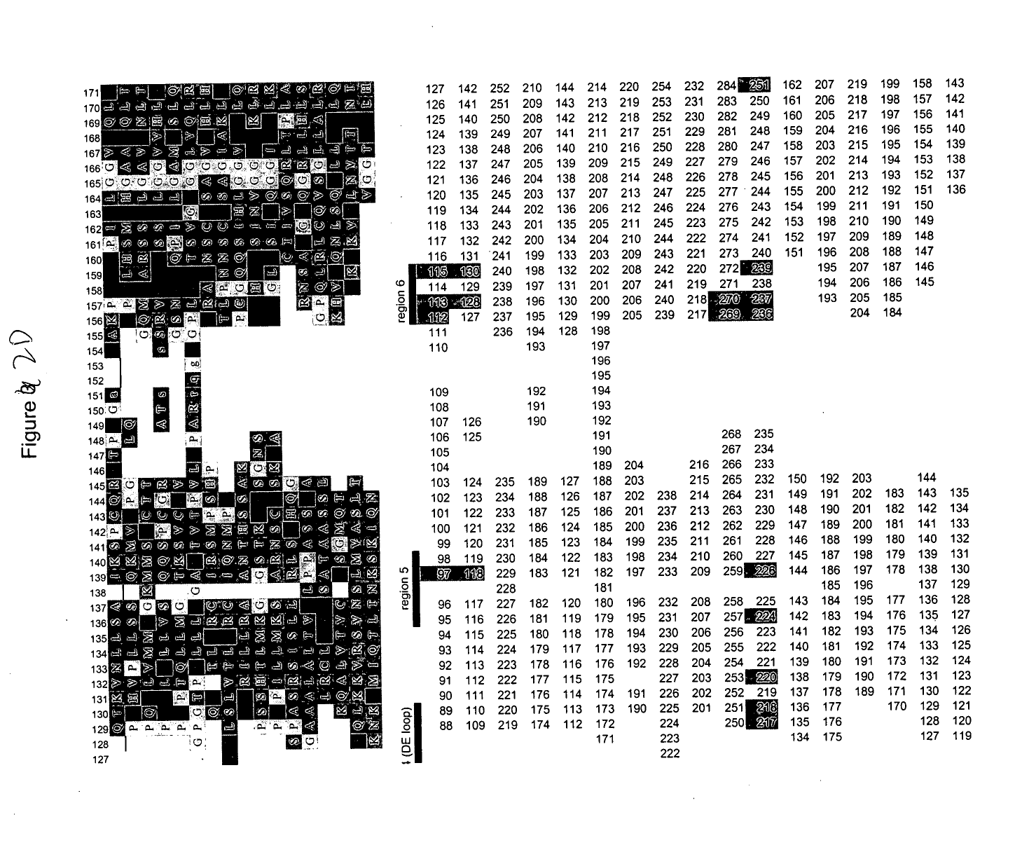

In a preferred embodiment, as highlighted in FIG. 2, the amino acid sequences of members of the TNF superfamily may be aligned into a multiple sequence alignment (MSA). The alignment shown in FIG. 2 was derived originally from the Pfam database, and then further manipulated according to structural alignment (using CE) of the crystal structures of TNFA, TNFB, CD40L, TRAIL, and BLyS. The MSA may also be used to extend the known structural information for additional recognized TNFSF members and other structural homologues and families. Due to the high extent of structural homology between different TNFSF members, the MSA may be used as a reliable predictor of the effects of modifications at various positions within the alignment. For this, the TNFA sequence and numbering shown in FIG. 2 can be used as an MSA reference point for any other TNFSF protein sequence. As used herein, referral to “TNFSF protein positions corresponding to TNFA amino acid X”, represents referral to a collection of equivalent positions in other recognized TNFSF members and structural homologues and families. For example, TNFSF protein positions corresponding to TNFA amino acid L75 corresponds to the following amino acid positions in the following TNFSF proteins: TNFA:L75, TNFB:Y96, FASL:P206, LIGHT:T161, VEGI:S99, Lymphotoxin beta:T158, APRIL:T177, BLyS:A207, CD40L:P188, RANKL:Q237, TRAIL:Q205, CD27L:S121, 4-1BBL:S162, TWEAK:Y176, CD30L:D157, OX40L:N114, AITRL:N106, and equivalent positions in other proteins recognized as TNFSF members.

For example, analysis of a structure of the complex of TNFB with the p55 (R1) receptor indicates that the amino acid Y108 in TNFB directly contacts the receptor. The analogous residue Y216 from TRAIL also directly contacts the DR5 receptor. The MSA thus predicts that the analogous residue 197 from TNFA also contacts a receptor. Consistent with this prediction, mutation of TNFA-197 to R or T results in a significant loss of receptor-binding affinity and biological signaling activity. The analysis for this contact position can be extended to all members of the family, predicting that the following positions are important for receptor interactions: FASL: Y218, LIGHT:Y173, VEGI:Y111, TNFC:Y170, APRIL:R189, BlyS:V219, CD40L:R200, RANKL:I249, CD27L:C133, 4-1BBL:A174, TWEAK:A188, CD30L:K169, OX40L:L126, and AITRL:Y118. This kind of analysis can be performed for all receptor contact regions of the ligands.

FIG. 2 highlights 7 canonical receptor contact regions based on analysis of known structures and mutational data. In preferred embodiments of the invention, each of the 7 regions highlighted in FIG. 2 as a receptor-contact region is used to define modification sites for the creation of variants of CD40L. In additional preferred embodiments, such modifications reduce receptor affinity and/or signaling capacity. In additional preferred embodiments, these modifications also preserve the ability of each protein to oligomerize with naturally occurring CD40L proteins, including, but not necessarily limited to, the corresponding wild-type sequence of each family member.

Using the alignment system depicted in FIG. 2 or other alignment programs discussed above, one can use as a reference point, the numbering system of any alignment program and may correlate the relevant positions of the TNFA protein with equivalent positions in other recognized members of the CD40L or structural homologues and families.

For purposes of the present invention, the areas of CD40L proteins to be modified are preferably, but not required to be, selected from the group consisting of the Large Domain, Small Domain, the DE loop, and the trimer interface. The Large Domain, the Small Domain and the DE loop are three separate receptor contact domains, each made up of several non-contiguous linear segments of the protein (i.e. the 7 canonical receptor contact regions described above). These domains are identified in the CD40L protein—and the MSA—by comparison to the receptor interaction domains of Lymphotoxin-alpha and TRAIL, two TNFSF proteins whose structures (PDB entries 1TNR and 1D0G, respectively) have been defined in complex with their cognate receptors using crystallographic methods. The trimer interface mediates interactions between individual CD40L protein monomers. Trimerization positions can be identified either directly from the crystal structure of the CD40L protein, or by analogy to another TNFSF protein. In a preferred embodiment, positions from one CD40L protein monomer containing atoms that are within 6 angstroms distance from a neighboring monomer are designated as trimer interface positions. Modifications may be made solely in one of these areas or in any combination of these and other areas.

The Large Domain preferred positions to be modified in CD40L proteins include but are not limited to TNFA corresponding positions 28-34, 63-69, 112-115, and 137-147. For the Small Domain, the preferred positions to be modified include but are not limited to TNFA corresponding positions 72-79 and 95-98. For the DE Loop, the preferred positions to be modified include but are not limited to TNFA corresponding positions 84-89. The Trimer Interface positions to be modified include but are not limited to TNFA corresponding positions 11, 13, 15, 34, 36, 53-55, 57, 59, 61, 63, 72, 73, 75, 77, 119, 87, 91-99, 102-104, 109,112-125, 147-149, 151, and 155-157. Especially preferred trimer interface positions to be modified are TNFA corresponding positions 57, 34, and 91. For example, amino acids X and Y at TNFA corresponding positions 34 and 91 can be replaced simultaneously by similarly charged amino acids (e.g. X34E+Y91 E, X34K+Y91R, etc.) to generate electrostatic repulsion at the variant monomer-monomer interfaces while not perturbing the stability of variant-native interfaces.

In a preferred embodiment, the choice of modification site and type is made by referring to other sequences in the alignment. Thus, in a preferred embodiment, the original amino acid X from sequence A is mutated to amino acid Y from sequence B, such that Y is a non-conservative substitution relative to amino acid X. For example, the amino acid Y87 from TNFA aligns with the non-conservative R189 from APRIL. Indeed, as previous studies have shown, the Y87R substitution in TNFA leads to a significant decrease in receptor binding and signaling by TNFA. In additional embodiments, more conservative mutations can also be utilized. In additional embodiments, the wild-type residue is mutated to alanine.

In another embodiment of the invention, receptor contact positions are predicted from a model of CD40L trimer complexed with a receptor. Using the crystal structure of CD40L (1ALY) and superposition of CD40L backbone coordinates (for a subset of positions according to the MSA) onto the backbone coordinates of TNF-β (from 1TNR), one obtains a model of CD40L complexed with the p55 TNF receptor. Although this is not a cognate receptor of CD40L, one can reasonably assume that CD40 will bind to CD40L in a similar manner. Analysis of the model structure indicates that the following CD40L residues are likely to be involved in receptor binding. For the purposes of the present invention, we define CD40L large domain receptor contact positions as I127, A130, E142, K143, G144, Y145, Y146, T176, C178, C218, Q220, S245, S248, H249, T251, F253, small domain receptor contact positions as S185, Q186, A187, P188, I190, R207, A209, S214, T242, and DE loop receptor contact positions as R200, F201, and R203. In a preferred embodiment, at least one of these amino acids is substituted with an alternative amino acid that modifies receptor binding and/or signaling. A number of these positions (K143, Y145, Y146, R203, R207, and Q220) have previously been shown with mutagenesis studies to be important for receptor interactions [see Singh et al., Protein Science 7:1124-1135 (1998), Bajorath et al., Biochemistry 34: 9884-9892 (1995), and Bajorath et al., Biochemistry 34: 1833-1844 (1995)]. In a preferred embodiment, receptor-disrupting mutations in at least two receptor contact domains (i.e. large domain, small domain, DE loop) are combined to reduce residual agonism of variant CD40L proteins.

In another embodiment of the invention, rational design of novel CD40L variants may be achieved by using, for example, Protein Design Automation® (PDA®) technology. (See U.S. Pat. Nos. 6,188,965; 6,269,312; 6,403,312; 6,708,120; 6,801,861; WO98/47089 and U.S. Ser. Nos. 09/652,699; 09/866,511; 09/990,769; 09/812,034; 09/837,886; 09/877,695; 10/057,552; 10/071,859; 10/888,748; 09/782,004; 09/927,790; 10/218,102; 10/218,102; 10/666,311; 10/666,307; and 60/602,546, filed Aug. 17, 2004, all references expressly incorporated herein in their entirety).

PDA® technology couples computational design algorithms that generate quality sequence diversity with experimental high-throughput screening to discover proteins with improved properties. The computational component uses atomic level scoring functions, side chain rotamer sampling, and advanced optimization methods to accurately capture the relationships between protein sequence, structure, and function. Calculations begin with the three-dimensional structure of the protein and a strategy to optimize one or more properties of the protein. PDAe technology then explores the sequence space comprising all pertinent amino acids (including unnatural amino acids, if desired) at the positions targeted for design. This is accomplished by sampling conformational states of allowed amino acids and scoring them using a parameterized and experimentally validated function that describes the physical and chemical forces governing protein structure. Powerful combinatorial search algorithms are then used to search through the initial sequence space, which may constitute 10⁵⁰ sequences or more, and quickly return a tractable number of sequences that are predicted to satisfy the design criteria. Useful modes of the technology span from combinatorial sequence design to prioritized selection of optimal single site substitutions. PDA® technology has been applied to numerous systems including important pharmaceutical and industrial proteins and has a demonstrated record of success in protein optimization.

PDA® utilizes three-dimensional structural information. For the purposes of the present invention, the crystal structure of CD40L (PDB accession 1ALY) can be used for PDA® simulations. Preferred substitutions at these positions are those predicted by PDA® technology to be structurally compatible with the CD40L trimer. Such substitutions include, but are not limited to, the following:

Mutations in the CD40L/CD40 interface positions disrupt receptor binding and activation. These positions can be identified by models of the CD40L/CD40 structure based on the structure of TNF bound to its receptor and the sequence homology between CD40L and TNF. These positions include, but are not limited to: 127, 130, 142, 143, 144, 145, 146, 176, 178, 184, 185, 186, 187, 188, 200, 201, 203, 207, 209, 218, 220, 242, 245, 248, 249, 251, 253.

The CD40L/CD40 interface substitutions that are compatible with the CD40L structure, being within 2.0 kcal/mole of lowest energy amino acid at each position include, but are not limited to, the following: I127H, I127V, I127Q, I127M, I127L, I127E, I127F, I127C, A130P, A130Q, A130S, A130K, A130M, A130N, A130T, E142Q, E142M, E142V, E142R, E142C, E142N, E142D, E142K, E142I, K143R, K143Q, G144Q, G144M, G144E, G144H, G144S, Y145P, Y145E, Y145H, Y145N, Y146H, Y146S, Y146C, T176A, T176S, T176G, C178G, C178S, C178A, C178D, C178E, C178H, C178Q, C178N, C178K, C178Y, C178M, S184T, S184E, S184Q, S184N, S184A, S184C, S184L, S184R, S184M, S184G, S184K, S185R, S185N, S185K, S185H, S185T, S185A, S185Q, Q186H, Q186K, Q186R, Q186N, Q186M, Q186E, Q186T, Q186D, Q186I, Q186V, Q186L, Q186S, Q186A, Q186C, A187E, A187H, A187W, A187Q, A187M, A187V, A187D, A187L, P188D, P188N, P188H, P188E, P188M, P188C, P188Q, P188K, P188T, P188A, P188V, P188S, I190V, I190Y, I190A, I190C, I190S, I190E, I190L, R200E, R200Q, R200H, R200D, R200N, R200M, R200S, F201R, F201H, F201E, F201N, F201Q, F201D, F201K, F201S, F201T, R203Y, R203E, R203F, R203H, R203Q, R203T, R203W, R203A, R203N, R203S, R203C, R203D, R207C, R207N, A209S, S214P, S214D, S214N, S214A, S214E, S214Q, C218P, Q220P, Q220E, Q220D, Q220M, Q220N, Q220C, Q220T, T242S, T242V, S245Q, S245R, S245P, S245E, S245N, S245H, S245K, S245D, S245M, S245T, S245V, S245A, S245L, S245G, S245Y, S245I, S248T, S248E, S248Q, S248C, S248A, H249E, H249Q, H249D, H249N, T251E, T251Q, T251M, T251D, T251P, T251V, T251N, T251H, T251I, T251A, T251L, T251S, T251R, T251G, F253E, F253Y, F253M, F253D, and F253H.

The CD40L/CD40 interface substitutions that are compatible with the CD40L structure, being within 2.0 kcal/mole of lowest energy amino acid at each position, and are also non-conservative mutations, as judged by BLOSUM62 scores less than 0.0 make good dominant negative mutations. These substitutions include, but are not limited to, the following:

I127H, I127Q, I127E, I127C, A130P, A130Q, A130K, A130M, A130N, E142M, E142V, E142C, E142I, G144Q, G144M, G144E, G144H, Y145P, Y145E, Y145N, Y146S, Y146C, T176G, C178G, C178S, C178D, C178E, C178H, C178Q, C178N, C178K, C178Y, C178M, S184C, S184L, S184R, S184M, S185R, S185H, Q186T, Q186I, Q186V, Q186L, Q186A, Q186C, A187E, A187H, A187W, A187Q, A187M, A187D, A187L, P188D, P188N, P188H, P188E, P188M, P188C, P188Q, P188K, P188T, P188A, P188V, P188S, I190Y, I190A, I190C, I190S, I190E, R200D, R200M, R200S, F201R, F201H, F201E, F201N, F201Q, F201D, F201K, F201S, F201T, R203Y, R203F, R203T, R203W, R203A, R203SS, R203D, R207C, S214P, C218P, Q220P, Q220C, Q220T, S245R, S245P, S245H, S245M, S245V, S245L, S245Y, S245I, S248C, H249D, T251E, T251Q, T251M, T251D, T251P, T251H, T251I, T251 L, F253E, F253D, F253H. Although the above mutations to Cys are generally unwanted because of their sulfur reactivity, they are particularly useful for modifying the protein with chemical moieties including PEG polymers.

Of the above dominant negative substitutions, especially favored substitutions are those that retain, or increase the polarity of the protein. These substitutions increase the solubility of the protein. The substitutions are to an amino acid that has a hydrophobicity of less than zero as defined by the scale of Faucher and Pliska. These especially favored substitutions include, but are not limited to: I127Q, I127E, I127S, I127D, A130Q, A130K, A130N, A130R, A130D, A130E, G144Q, G144E, G144K, G144R, Y145E, Y145N, Y145D, Y146S, Y146N, C178S, C178D, C178E, C178Q, C178N, C178K, C178R, S184R, S185R, A187E, A187Q, A187D, A187N, A187K, A187R, P188D, P188N, P188E, P188Q, P188K, P188S, I190S, I190E, R200D, R200S, F201R, F201E, F201N, F201Q, F201D, F201K, F201S, R203S, R203D, S214R, C218D, S245R, H249D, T251E, T251Q, T251D, T251R, F253E, and F253D.

In a preferred embodiment, suitable substitutions are defined as those that are observed at homologous positions in proteins that are CD40L homologues. For example, comparison to the MSA indicates that other TNFSF proteins frequently contain T, S, and Q at positions equivalent to P188. In additional embodiments, more dramatic effects on receptor interactions are expected when non-conservative substitutions are made, such as when residues such as D, E, K, and R are replaced with residues of opposite charge (e.g. D and E→K or R, K and R→D or E). For example, the predicted receptor contact residues K143, R200, R203, R207 can be replaced with D or E to create variants with reduced receptor interactions. Likewise, E142 can be substituted with K or R to create variants with reduced receptor interactions. Indeed, as shown by Singh et al., Protein Science 7: 1124-1135 (1998), the substitutions K143E, R203E, and R207E all lead to significant loss of receptor binding.

Additional useful positions for modifying receptor-binding properties of CD40L variants can be discovered by inspection of mutations associated with the X-linked form of hyper-IgM syndrome. Patients with this syndrome have mutations, found in the gene encoding CD40L (located on the X chromosome), which disrupt the normal function of CD40L. According to CD40Lbase (uta.fi/imt/bioinfo/CD40Lbase), a publicly available database of CD40L mutations found in hyper-IgM patients, these mutations include, but are not limited to: M36R, K96N, A123E, H125R, V126D, V126A, S128R, E129G, W140C, W140G, W140R, W140X, E142X/K143X, G144E, Y145X, T147N, L155P, Y169D, Y170C, Y170P, A173D, Q174R, T176I, A183@, S184X, Q186X, L193@, L195P, R200X, E202X, R203I, A208D, C218X, Q220X, Q221X, H224Y, G226A, G227V, L231S, Q232X, A235P, S236X, V237E, T254M, G257D, G257S, and L258S, where X denotes a deletion of the amino acid and @ denotes insertions of one or more amino acids at these locations. (See also, Lee et al. Blood. 2004 Eprint, 9 Sep. 2004.) As will be appreciated by those in the art, additional substitutions at these positions are also likely to impact the structure and/or function of CD40L. While these mutations are expected to have a variety of effects on the structure and receptor-binding properties of CD40L, a number of them map directly to the predicted receptor-interaction domains outlined herein, suggesting that they disrupt the function of CD40L by directly affecting receptor binding rather than by destabilizing the tertiary and quaternary structure of CD40L. Preferred mutations from this subset therefore include C218X, S128R, E129G, E142X/K143X, K143T, G144E, Y145X, Y145N, T147N, T176I, R203I, A208D, Q220X, and T254M. As will be appreciated by those in the art, multiple alternative substitutions at these positions are also expected to affect receptor binding.

Many of the hyper-IgM mutations sites are found to be directly in the interface of CD40L and its receptor. The appropriate amino acid substitutions that are energetically favorable by the PDA® technology, as described previously, and are disruptive to receptor binding include, but are not limited to: E142M, E142V, E142C, E142I, G144Q, G144M, G144E, G144H, Y145P, Y145E, Y145N, T176G, R203Y, R203F, R203T, R203W, R203A, R203S, R203D, R207C, C218P, Q220P, Q220C, and Q220T.

Other hyper-IgM mutation sites are found immediately adjacent to the interface sites previously identify and would also reduce CD40L/CD40 binding. Substitutions at these sites that are within 2.0 kcal/mole of the lowest energy amino acid at the site, as judged by PDA® technology and are disruptive, as judged by BLOSUM62 scores less than zero include, but are not limited to: S128C, E129V, K143A, K143T, T147I, C218P, C218G, C218D.

CD40L binds the antibody, 5c8. This antibody is known to compete with CD40 for binding to CD40L (Karpusas et al. 2001 Structure, 9:312-329). The surface on CD40L that binds the antibody is largely the same surface that binds to CD40 and mutations disrupting 5c8 binding will also reduce CD40 binding. Therefore, dominant negative mutations can be designed based on the CD40 surface that binds the antibody. The residues that comprise the antibody-binding site on CD40L include, but are not limited to: E129, A130, S131, S132, A141, E142, K143, G144, Y146, C178, S179, P217, C218, Q220, S245, Q246, V247, S248, H249, G250, and T251.

Stable mutations at these sites that disrupt receptor binding are those that are low in energy, within 2.5 kcal/mole of the lowest energy amino acid as judged by PDA™ technology, and are non-conservative, based on BLOSUM62 scores less than zero. These substitutions include, but are not limited to: E129V, A130P, A130Q, A130K, A130M, A130N, A130H, A130R, A130L, A130D, A130E, S132R, S132H, S132P, S132M, S132V, S132L, S132W, S132I, E142M, E142V, E142C, E142I, E142T, E142A, K143A, K143T, G144Q, G144M, G144E, G144H, G144C, G144K, G144R, Y146S, Y146C, Y146A, Y146N, C178G, C178S, C178D, C178E, C178H, C178Q, C178N, C178K, C178Y, C178M, C178R, C178F, C178W, S179C, P217D, P217E, P217Y, P217Q, P217H, P217S, P217F, P217N, P217A, P217G, P217V, P217T, P217L, C218P, C218G, C218D, Q220P, Q220C, Q220T, Q220V, Q220A, Q220I, S245R, S245P, S245H, S245M, S245V, S245L, S245Y, S245I, S245C, S245F, S245W, Q246A, Q246C, Q246G, V247C, S248C, H249D, H249V, T251E, T251Q, T251M, T251D, T251P, T251H, T251I, T251L, T251R, T251G, and T251C.

Especially preferred substitutions in the above list are those that maintain or increase the solubility of the protein by increasing its polarity. These substitutions are to amino acids that have Faucher and Pliska hydrophobicity values less than 0.0. Mutations that disrupt the receptor/antibody binding and increase the polarity of the protein include, but are not limited to: A130Q, A130K, A130N, A130R, A130D, A130E, S132R, G144Q, G144E, G144K, G144R, Y146S, Y146N, C178S, C178D, C178E, C178Q, C178N, C178K, C178R, P217D, P217E, P217Q, P217S, P217N, C218D, E129V, A130P, A130Q, A130K, A130M, A130N, A130H, A130R, A130L, A130D, A130E, S132R, S132H, S132P, S132M, S132V, S132L, S132W, S132I, E142M, E142V, E142C, E142I, E142T, E142A, K143A, K143T, G144Q, G144M, G144E, G144H, G144C, G144K, G144R, Y146S, Y146C, Y146A, Y146N, C178G, C178S, C178D, C178E, C178H, C178Q, C178N, C178K, C178Y, C178M, C178R, C178F, C178W, S179C, P217D, P217E, P217Y, P217Q, P217H, P217S, P217F, P217N, P217A, P217G, P217V, P217T, P217L, C218P, C218G, C218D, Q220P, Q220C, Q220T, Q220V, Q220A, Q220I, S245R, S245P, S245H, S245M, S245V, S245L, S245Y, S245I, S245C, S245F, S245W, Q246A, Q246C, Q246G, V247C, S248C, H249D, H249V, T251E, T251Q, T251M, T251D, T251P, T251H, T251I, T251L, T251R, T251G, and T251C.

Analysis of the CD40L crystal structure indicates that trimer interface residues of CD40L include the following: Q118, N119, Q121, A123, H125, Y145, T147, S149, Q166, G167, L168, Y170, Y172, Q174, T176, R181, P188, I190, L195, R203, I204, L205, L206, R207, A208, A209, N210, T211, S213, S214, A215, K216, P217, G219, Q220, Q221, S222, I223, H224, L225, G226, G227, V228, F229, E230, Q232, G250, T251, G252, F253, S255, L259, and L261. In a preferred embodiment, one or more of these residues are substituted with alternative amino acids in order to modify trimerization properties of CD40L variants. Trimerization properties that could be modified include, but are not limited to, kinetics of exchange with native CD40L monomers, heterotrimer stability, and homotrimer stability. The substitutions at these trimer interface positions that are energetically favorable, being within 2.0 kcal/mol in stability to the lowest energy amino acid at each site, include, but are note limited to: Q118H, Q118E, Q118Q, Q118N, Q118A, Q118T, Q188S, Q118W, Q118F, Q118V, Q118G, Q118C, Q118K, Q118R, Q118Y, Q118D, Q118M, N119H, N119Q, N119L, N119N, N119R, N119D, N19A, N119S, N119M, N119K, N119G, N119E, Q121H, Q121Q, Q121T, Q121V, Q121Y, Q121M, Q121C, Q121D, Q121A, Q121E, Q121I, Q121N, Q121F, Q121L, Q121S, Q121G, A123A, H125H, H125N, H125M, Y145P, Y145E, Y145Y, Y145H, Y145N, T147V, T147T, T147I, S149S, S149A, Q166S, Q166G, Q166C, Q166D, Q166Q, Q166E, Q166A, Q166N, G167G, L168L, L168P, Y170Y, Y172Y, Q174I, Q174Q, Q174V, Q174L, Q174H, Q174K, T176A, T176S, T176G, T176T, R181G, R181S, R181C, R181A, S185R, S185S, S185N, S185K, S185H, S185T, S185A, S185Q, Q186H, Q186K, Q186R, Q186Q, Q186N, Q186M, Q186E, Q186T, Q186D, Q186I, Q186V, Q186L, Q186S, Q186A, Q186C, P188P, P188D, P188N, P188H, P188E, P188M, P188C, P188Q, P188K, P188T, P188A, P188V, P188S, I190V, I190I, I190Y, I190A, I190C, I190S, I190E, I190L, L195L, R200E, R200Q, R200H, R200D, R200N, R200M, R200S, R203Y, R203E, R203F, R203H, R203Q, R203T, R203W, R203A, R203N, R203S, R203R, R203C, R203D, I204E, I204V, I204P, I204L, I204M, I204I, I204T, I204Q, I204C, L205L, L206Q, L206L, L206M, L206E, R207R, R207C, R207N, A208A, A209A, A209S, N210Q, N210N, N210C, N210E, T211D, T211Y, T211E, S213S, S213D, S213A, S214S, S214P, S214D, S214N, S214A, S214E, S214Q, A215G, A215A, A215S, K216D, P217P, P217D, P217E, P217Y, P217Q, P217H, P217S, P217F, P217N, P217A, P217G, C218P, G219G, Q220P, Q220E, Q220D, Q220M, Q220Q, Q220N, Q220C, Q220T, Q221L, Q221Y, Q221M, Q221E, Q221S, Q221D, Q221F, S222S, S222A, S222T, S222C, I223I, H224F, H224H, L225L, G226G, G227G, V228V, F229F, E230P, E230H, E230M, E230E, E230Q, Q232D, Q232E, Q232H, Q232N, Q232Q, Q232Y, Q232V, Q232S, G250G, T251E, T251Q, T251M, T251D, T251P, T251V, T251N, T251T, T251H, T251I, T251A, T251L, T251S, T251R, T251G, G252G, F253F, F253E, F253Y, F253M, F253D, F253H, S255S, S255V, S255C, S255T, S255A, L259L, K260S, K260K, K260A, K260C, K260Q, K260M, K260N, K260E, L261V, L261L, L261I, L261T, L261C, L261A.

In a preferred embodiment, amino acid substitutions, deletions, or insertions that influence the kinetics of exchange between variant and wild-type monomers are made either individually or in combination. These substitutions may also be combined with additional substitutions that affect receptor interaction or other properties. Substitutions that have an effect on exchange properties may include substitutions at positions F201, I204, and other DE loop positions, and A209, among others.

Soluble CD40L Variants

In a preferred embodiment, PDA® technology is utilized for the rational design of CD40L variants that are expressed as soluble proteins, in host cell systems including, prokaryotes and eukaryotes, with preferred embodiments expressing in bacterial host cell systems. Furthermore, the generation of soluble CD40L variants enables the development of CD40L variants with novel biological properties, including but not limited to CD40L variants that act as CD40L antagonists.

A variety of strategies may be utilized to design CD40L variants that express solubly in E. coli. In a preferred embodiment, three strategies are used: 1) reduce hydrophobicity by replacing solvent-exposed hydrophobic residues with suitable polar residues, 2) increase polar character by replacing neutral polar residues with charged polar residues 3) replace non-disulfide bonded cysteine residues (unpaired cysteines) with suitable non-cysteine residues, and 4) replace residues whose identity is different in murine versus human CD40L. As will be appreciated by those in the art, several alternative strategies could also be utilized. For example, modifications that increase the stability of a protein can sometimes improve solubility by decreasing the population of partially folded or misfolded states. As another example, protein solubility is typically at a minimum when the isoelectric point of the protein is equal to the pH of the surrounding solution. Modifications, which perturb the isoelectric point of the protein away from the pH of a relevant environment, such as serum, can therefore serve to improve solubility.

Replacing Solvent-Exposed Hydrophobic Residues with Suitable Polar Residues

In a preferred embodiment, solvent exposed hydrophobic residues are replaced with structurally and functionally compatible polar residues. Alanine and glycine may also serve as suitable replacements, constituting a reduction in hydrophobicity. Solvent exposed hydrophobic residues can be defined according to absolute or fractional solvent accessibility, as defined above. Examples of solvent exposed hydrophobic residues in CD40L are 120, 127, 145, 146, 152, 178, 201, 204, 217, 218, 233, 261. It is also possible to use other methods, such as contact models, to identify exposed residues. Especially preferred solvent exposed hydrophobic residues are those residues that have not been implicated in mediating CD40L function.

In an alternate embodiment, preferred polar residues include those that are observed at homologous positions in proteins that are CD40L homologues. In a most preferred embodiment, the CD40L homologues comprise mouse CD40L. Additional preferred CD40L homologues include allelic variants of the human CD40L and CD40L other related species. Alternatively, CD40L homologues may comprise other TNF superfamily members including but not limited to TNF-alpha, LT, TRAIL, BAFF, and CD40L. See also U.S. Ser. No. 10/338,083, filed on Jan. 6, 2003, hereby incorporated by reference in its entirety.

In an especially preferred embodiment, suitable polar residues include only the subset of polar residues with low or favorable energies as determined using PDA® technology calculations. For example, suitable polar residues may be defined as those polar residues whose energy in the optimal rotameric configuration is more favorable than the energy of the exposed hydrophobic residue observed in the wild-type protein at that position, or those polar residues whose energy in the optimal rotameric configuration is among the most favorable of the set of energies of all polar residues at that position. Preferred polar residues include, but are not limited to, glutamine, glutamic acid, lysine, and arginine.

In the most preferred embodiment, suitable polar residues include the subset of polar residues that are deemed suitable by both PDAe calculations and by sequence alignment data.

Some substitutions are favorable for increasing the solubility of CD40L. These substitutions minimize the exposure of hydrophobic atoms. The exposure of hydrophobic residues is judged by the product of the fraction accessibility of a residue, from zero to one, times the hydrophobicity of the residue, as measured by the scale of Faucher and Pliska (Faucher and Pliska, 1983 Eur. J. Med. Chem., 18, 369-375). Some residues exposed a significant amount of exposed hydrophobic surface. That is, their non-polar exposure product (NEP) was greater than 0.2. These positions include, but are not limited to: 120, 127, 134, 137, 145, 146, 148, 152, 155, 178, 183, 188, 190, 198, 201, 204, 217, 218, 233, and 261.

To mitigate the tendency of CD40L to aggregate, the wild type amino acid at each position should be changed to an amino acid that (1) fits well into the structure to avoid misfolding or destabilization of the native structure, and (2) is polar enough to avoid hydrophobic contacts with other non-polar constituents. Our designed mutants that satisfy criterion (1) are those that have energies within 2.0 kcal/mole of the most favorable amino acid at that position as determined by PDA® algorithms. These substitutions include, but are not limited to:

P120H, P120R, P120Q, P120N, P120E, P120D, P120K, P120Y, P120V, P120F, P120A, P120G, P120T, P120S, P120M, P120C, P120I, P120L, I127H, I127V, I127Q, I127M, I127L, I127E, I127F, I127C, I127T, I127A, I127S, I127D, T134Q, T134E, T134D, T134R, T134N, T134H, T134K, T134P, T134S, T134M, T134G, T134V, T134A, T134L, T134I, T134Y, V137H, V137R, V137I, V137T, V137C, V137A, V137M, V137K, V137S, Y145P, Y145E, Y145H, Y145N, Y145D, Y146H, Y146S, Y146C, Y146A, Y146N, M148K, L152E, L152Q, C178G, C178S, C178A, C178D, C178E, C178H, C178Q, C178N, C178K, C178Y, C178M, C178R, C178F, C178W, A183Q, A183E, A183H, A183R, A183D, A183N, A183K, A183S, A183T, A183L, A183V, A183G, A183M, A183I, A183C, A183Y, P188D, P188N, P188H, P188E, P188M, P188C, P188Q, P188K, P188T, P188A, P188V, P188S, P188F, P188G, I190V, I190Y, I190A, I190C, I190S, I190E, I190L, I190T, I190F, I190M, P198E, P198D, P198N, P198V, P198Q, P198T, F201R, F201H, F201E, F201N, F201Q, F201D, F201K, F201S, F201T, F201P, F201M, F201V, F201G, F201C, F201A, I204E, I204V, I204P, I204L, I204M, I204T, I204Q, I204C, I204A, I204S, P217D, P217E, P217Y, P217Q, P217H, P217S, P217F, P217N, P217A, P217G, P217V, P217T, P217L, C218P, C218G, C218D, P233E, P233D, P233S, P233Q, P233C, P233N, P233T, P233V, P233M, P233A, P233H, P233R, P233G, L261V, L261I, L261T, L261C, L261A, L261N, L261S, L261D.

Substitutions satisfying both these criteria, being energetically favorable for the position and substituting in a polar amino acid containing a negative Faucher and Pliska hydrophobicity score, include but are not limited to: P120R, P120Q, P120N, P120E, P120D, P120K, P120G, P120S, I127Q, I127E, I127S, I127D, T134Q, T134E, T134D, T134R, T134N, T134K, T134S, T134G, V137R, V137K, V137S, Y145E, Y145N, Y145D, Y146S, Y146N, M148K, L152E, L152Q, C178G, C178S, C178D, C178E, C178Q, C178N, C178K, C178R, A183Q, A183E, A183R, A183D, A183N, A183K, A183S, A183G, P188D, P188N, P188E, P188Q, P188K, P188S, P188G, I190S, I190E, P198E, P198D, P198N, P198Q, F201R, F201E, F201N, F201Q, F201D, F201K, F201S, F201G, I204E, I204Q, I204S, P217D, P217E, P217Q, P217S, P217N, P217G, C218G, C218D, P233E, P233D, P233S, P233Q, P233N, P233R, P233G, L261N, L261S, and L261D.

F201 is particularly exposed for a hydrophobic amino acid, having 95% of its surface area exposed and the highest non-polar exposure product (NEP) of 1.7. Many substitutions at this position will reduce the exposed hydrophobic surface and this is an important site to change for increasing solubility. Therefore, the substitutions at F201 which reduce the exposure of non-polar surface area include, but are not limited to: F201R, F201E, F201N, F201Q, F201D, F201K, F201S, F201H, F201T, F201A and F201G.

Replacing Non-Disulfide Bonded Cysteine Residues with Suitable Non-Cysteine Residues

In another preferred embodiment, free cysteine residues (that is, cysteine residues that are not participating in disulfide bonds) are mutated to a structurally and functionally compatible non-cysteine residue. Unpaired cysteines can be identified by visual analysis of the structure or by analysis of the disulfide bond patterns of related proteins. Human CD40L contains a single unpaired cysteine at position 194. PDA® simulations indicate that a C194A substitution would have the least impact on the stability and function of CD40L trimers. Alternative favorable substitutions predicted by PDA® simulations include, but are not limited to C194Q, C194K, and C194M.

In a preferred embodiment, suitable residues are defined as those with low (favorable) energies as calculated using PDA® technology. For example, suitable residues may be defined as those non-cysteine residues whose energy in the optimal rotameric configuration is more favorable than the energy of the cysteine residue observed in the wild-type protein at that position, or those residues whose energy in the optimal rotameric configuration is among the most favorable of the set of energies of all residues at that position. In a preferred embodiment, suitable residues are defined as those that are observed at homologous positions in proteins that are CD40L homologues. For example, comparison to the MSA indicates that other TNFSF proteins frequently contain T, S, Y, and Q at positions equivalent to C194. In a more preferred embodiment, suitable substitutions are those such as C194Q, which have both low (favorable) energies as calculated using PDA® technology and are observed at homologous positions in proteins that are CD40L homologues.

Replacing Disulfide Bonded Cysteine Residues with Suitable Non-Cysteine Residues

Inspection of the CD40L crystal structure reveals a disulfide bond formed between cysteine residues C178 and C218. Compared to most disulfides observed in natural protein structures, this disulfide is considerably solvent exposed, and is anticipated to be labile. Therefore, in some embodiments of the invention, this pair of cysteine residues will be replaced by alternative amino acids in order to prevent issues associated with chemical crossreactivity, for example when attaching a PEG moiety to confer improved pharmacokinetic properties onto variant CD40L proteins. PDA® simulations indicate that suitable replacements include, but are not limited to, the following: C178S/C218E, C178S/C218Q, C178T/C218E, C178T/C218Q, C178A/C218E, and C178A/C218Q.

Reduction of Alpha IIb beta3 Integrin Binding and/or Activation

In order to minimize risk of platelet activation, variant CD40L proteins of the invention preferably have reduced or eliminated interactions with alpha IIb beta3 integrin. This interaction appears to be mediated by the KGD motif in positions 115 to 117 of CD40L, which is recognized by the RGD recognition domains of the integrin. The KGD motif is, however, not contained within the TNF homology domain of CD40L, suggesting that its removal will have no impact on the structural integrity and antagonist activity of CD40L variants. In preferred embodiments, the KGD motif is removed by deletion of one or more of the KGD amino acids (e.g. KGD→XGD, KXD, or KGX), mutation of one or more of the KGD amino acids (e.g. KGD→KGE, KGD→EGD, etc.), or chemical modification of one or more of the KGD amino acids (e.g. PEGylation at K).

Dominant Negative CD40L Variants

In a preferred embodiment, CD40L variants are engineered to yield significantly reduced affinity and/or signaling for CD40L receptors relative to wild-type CD40L while maintaining affinity for other CD40L proteins to allow formation of mixed trimers. Such CD40L variants are referred to as “dominant negative CD40L variants” or “DN-CD40L”. The dominant negative CD40L variants act by sequestering the naturally occurring CD40L proteins in mixed heterotrimers that are incapable of appreciably activating the CD40L receptors. Consequently, DN-CD40L act to antagonize the action of naturally occurring CD40L. Alternatively, the amount of variant homotrimers “swamps” out the effect of endogenous homotrimers.

In a preferred embodiment, DN-CD40L variant proteins exhibit decreased biological activity as compared to wild-type CD40L, including but not limited to, decreased binding to the either CD40 and/or alpha IIb-beta3 integrin receptors.

In an alternate preferred embodiment, DN-CD40L variant proteins do not bind to either CD40 or alpha IIb-beta3 integrin receptors.

Variant CD40L proteins that exhibit less than 50% biological activity as compared to wild-type are preferred. More preferred are variant CD40L proteins that exhibit less than 25%, even more preferred are variant proteins that exhibit less than 15%, and most preferred are variant CD40L proteins that exhibit less than 10% of a biological activity of wild-type CD40L. Suitable assays are discussed further below.

Thus, the invention provides variant CD40L proteins with altered binding affinities such that the DN-CD40L proteins will preferentially oligomerize with wild-type CD40L, but do not substantially interact with wild-type CD40 or alpha IIb-beta3 integrin receptors. “Preferentially” in this case means that given equal amounts of variant CD40L monomers and wild-type CD40L monomers, at least 25% of the resulting trimers are mixed trimers of variant and wild-type CD40L, with at least about 50% being preferred, and at least about 80-90% being particularly preferred. In other words, it is preferable that the affinity of DN-CD40L variants for wild-type CD40L is greater than the affinity of a DN-CD40L variant for another DN-CD40L protein or than the affinity of wild-type CD40L for another wild-type CD40L protein.

For purposes of the present invention, the areas of the wild-type or naturally occurring CD40L molecule to be modified are preferably (but not required to be) selected from the group consisting of the Large Domain (also known as II), Small Domain (also known as I), the DE loop, and the trimer interface.

Modifications to Large Domain, Small Domain, or DE loop positions are expected to have direct effects on receptor binding and/or signaling. As will be appreciated by those in the art, additional modifications outside of these domains can also indirectly affect receptor binding and/or signaling.

Modifications at the trimer interface can be engineered to optimize the ability of CD40L variants to hetero-trimerize with wild-type CD40L proteins or to increase kinetics of exchange with native CD40L. In some cases, this can be accomplished with a single substitution at one trimer interface position. In other cases, two or more substitutions at multiple trimer interface positions must be combined.

In a preferred embodiment, substitutions, insertions, deletions or other modifications at multiple receptor interaction (i.e. large domain, small domain, DE loop) and/or trimerization domains may be combined. Such combinations are frequently advantageous in that they have additive or synergistic effects on dominant-negative activity and reduced propensity to activate CD40 signaling. Examples include, but are not limited to, simultaneous substitution of amino acids at the large and small domains (e.g. Q220T+R203D, Y145N+P188N), large domain and DE loop (e.g. Y145D+F201S, G144Q+R200D), large domain and trimerization domain, or multiple substitutions within a single domain (G144Q+Y145N). Additional examples include any and all combinations of substitutions. Mutations that effect receptor binding can be combined with mutations that increase solubility (e.g. G144K+F201S). Mutations occurring naturally in hyper-IgM patients may be combined with any other mutation, such as a dominant negative mutation (e.g. T254M+Y146S). The total number of mutations is not limited, although the preferred number is generally less than or equal to 5 and, more preferably, less than or equal to 3.

In a most preferred embodiment, modifications at receptor interaction and/or trimerization domains are combined with CD40L substitutions that reduce interaction with alpha IIb beta3 integrin as described above.

In a preferred embodiment, CD40L variants may exchange with and attenuate the signaling potency of soluble CD40L.

In another embodiment, other members of the TNF superfamily (TNFSF) such as TNF-α, lymphotoxin-α, lymphotoxin-β, Fas ligand (FasL), TRAIL, CD30 ligand, CD27 ligand, Ox40 ligand, APRIL, BLyS, 4-IBBL, TRANCE and RANKL (OPGL), and any other protein that is recognized to be a member of the TNFSF, may be modified to form heterotrimers with native CD40L. In addition, CD40L variants may be designed to interact with other TNFSF members to achieve mixed heterotrimers. See, U.S. Ser. No. 10/338,083, filed Jan. 6, 2003 and its continuation-in-part, filed Jul. 7, 2003, Litinskiy et al, Nature Immunology, 3(9): 822 (September 2002); both incorporated by reference in their entirety.

Single Chain Dominant Negative CD40L Variants

An additional embodiment includes linked dimers of the CD40L variants. The present invention relates to these single chain polypeptides comprising multiple receptor-interaction domains that are modified such that each domain has significantly reduced affinity for the cognate receptor(s). Such linked domains preferably retain association with individual monomer domains such that they will exhibit a dominant-negative phenotype, antagonizing the action of the free monomer domains.

CD40L Proteins as Competitive Inhibitors

In an alternative embodiment, CD40L variants are engineered to yield monomers, dimers, or trimers that bind to the CD40L receptors but do not appreciably activate the CD40L receptors. These variants compete with naturally occurring CD40L protein for binding to the CD40L receptors, thereby limiting the ability of naturally occurring CD40L to bind and activate the CD40L receptors. Such CD40L variants are referred to as “competitive inhibitor CD40L variants” or “ciCD40L”.

In a preferred embodiment, ciCD40L comprises two variant CD40L monomers that are covalently connected. Such a construct would block wild-type CD40L from binding two of the three subunits that form the CD40L receptors. Furthermore, the affinity of a dimeric ciCD40L would likely be higher than an equivalent monomeric ciCD40L, facilitating competition. Linkers include, but are not limited to, polypeptide linkages between N- and C-termini of the domains, linkage via a disulfide bond between monomers, and linkage via chemical cross-linking reagents. Alternatively, the N- and C-termini may be covalently joined by deletion of portions of the N- and/or C-termini and linking the remaining fragments via a linker or linking the fragments directly.

In a preferred embodiment, ciCD40L variant proteins exhibit decreased biological activity as compared to wild-type CD40L, including but not limited to, decreased binding to alpha IIb-beta3 integrin, and/or decreased activation of CD40. Suitable assays include, but are not limited to, those described below. Furthermore, ciCD40L proteins are capable of inhibiting the biological functioning of wild-type CD40L.

Variant CD40L proteins that reduce the biological activity of wild-type CD40L by at least 50% are preferred. More preferred are variant CD40L proteins that reduce the biological activity of wild-type CD40L by 75%. Especially preferred ciCD40L variants reduce the activity of wild-type CD40L by at least 90%.

Thus, the invention provides variant CD40L proteins with altered binding affinities such that the ciCD40L proteins will form monomers, dimers, or trimers, and will bind to the CD40L receptors without signaling. In a preferred embodiment, the affinity of ciCD40L for the CD40L receptors is greater than the affinity of wild-type CD40L for the CD40L receptors. It is especially preferred that ciCD40L binds to CD40 with at least 10-fold greater affinity than the wild-type CD40L.

For purposes of the present invention, the areas of the wild-type or naturally occurring CD40L molecule to be modified are selected from the group consisting of the Large Domain (also known as II), Small Domain (also known as I), the DE loop, and the trimer interface. The Large Domain, the Small Domain and the DE loop are three separate receptor contact domains, each made up of several non-contiguous linear segments of the protein. These domains are identified in the CD40L protein by comparison to the receptor interaction domains of Lymphotoxin-alpha and TRAIL, two TNF superfamily homologues of CD40L whose structures (PDB accession codes 1TNR and 1 DOG, respectively) have been defined in complex with their cognate receptors using crystallographic methods. The trimer interface mediates physical interactions between CD40L monomers. Trimerization positions can be identified directly from the crystal structure of the mouse CD40L protein. In a preferred embodiment, positions from one CD40L monomer containing atoms that are within 5 angstroms distance from a neighboring CD40L monomer are designated as trimer interface positions. Modifications may be made solely in one of these areas or in any combination of these and other areas.

CD40L Superagonist Variants

In a preferred embodiment, PDA® technology is utilized for the rational design of CD40L variants that behave as superagonists. CD40L superagonist variants are CD40L variants that bind to and/or activate the CD40L receptors more strongly than the wild-type human CD40L does. As a result, CD40L superagonists could be used as a treatment for CD40L-responsive conditions that result from insufficient activity of endogenous CD40L.

In a preferred embodiment, CD40L superagonists exhibit increased biological activity as compared to wild-type CD40L, including but not limited to increased binding to CD40L receptors, increased activation of the CD40L receptors, and/or increase in cytotoxic activity. In a preferred embodiment the CD40L superagonist proteins are at least 10% more active then wild-type human CD40L, with at least 50%, 100% or 200% increases in activity being especially preferred. Suitable assays are discussed further below.

For purposes of the present invention, the areas of the wild-type or naturally occurring CD40L molecule to be modified are selected from the group consisting of the Large Domain (also known as II), Small Domain (also known as I), the DE loop, and the trimer interface. The Large Domain, the Small Domain and the DE loop are three separate receptor contact domains, each made up of several non-contiguous linear segments of the protein.

Generation of Nucleic Acids Encodinq CD40L Variants

In a preferred embodiment, nucleic acids encoding CD40L variants are prepared by total gene synthesis, or by site-directed mutagenesis of the DNA encoding wild-type or variant CD40L protein. Methods including template-directed ligation, recursive PCR, cassette mutagenesis, site-directed mutagenesis or other techniques that are well known in the art may be utilized.

Using the nucleic acids of the present invention, which encode a variant CD40L protein, a variety of expression vectors can be made. Preferred bacterial expression vectors include but are not limited to pET, PBAD, bluescript, pUC, pQE, pGEX, pMAL, and the like. Generally, these expression vectors include transcriptional and translational regulatory nucleic acid operably linked to the nucleic acid encoding the variant CD40L protein. Transcriptional and translational regulatory sequences may include, but are not limited to, promoter sequences, ribosomal binding sites, transcriptional start and stop sequences, translational start and stop sequences, and enhancer or activator sequences. Furthermore, the vector will typically include a selectable marker such as an antibiotic resistance gene.

A suitable bacterial promoter is any nucleic acid sequence capable of binding bacterial RNA polymerase and initiating the downstream (3′) transcription of the coding sequence of the variant CD40L protein into mRNA. A bacterial promoter has a transcription initiation region, which is usually placed proximal to the 5′ end of the coding sequence. This transcription initiation region typically includes an RNA polymerase binding site and a transcription initiation site. Sequences encoding metabolic pathway enzymes provide particularly useful promoter sequences. Examples include promoter sequences derived from sugar metabolizing enzymes, such as galactose, lactose and maltose, and sequences derived from biosynthetic enzymes such as tryptophan. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter may include naturally occurring promoters of non-bacterial origin that have the ability to bind bacterial RNA polymerase and initiate transcription.

In addition to a functioning promoter sequence, an efficient ribosome binding site is desirable. In E. coli, the ribosome binding site is called the Shine-Delgarno (SD) sequence and includes an initiation codon and a sequence 3-9 nucleotides in length located 3-11 nucleotides upstream of the initiation codon.

The expression vector may also include a signal peptide sequence that provides for secretion of the variant CD40L protein in bacteria. The signal sequence typically encodes a signal peptide comprised of hydrophobic amino acids which direct the secretion of the protein from the cell, as is well known in the art. The protein is either secreted into the growth media (gram-positive bacteria) or into the periplasmic space, located between the inner and outer membrane of the cell (gram-negative bacteria). For expression in bacteria, usually bacterial secretory leader sequences, operably linked to a variant CD40L encoding nucleic acid, are preferred.

The bacterial expression vector may also include a selectable marker gene to allow for the selection of bacterial strains that have been transformed. Suitable selection genes include genes, which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways.

Suitable expression vectors for non-bacterial expression systems are also well known in the art and can be utilized.

In an alternate embodiment, variant CD40L proteins or fragments thereof may be prepared by chemical synthesis. In such an embodiment, it is not necessary to generate nucleic acids encoding the CD40L variants.

Deimmunizing CD40L Variants

CD40L variants may be assessed for immunogenicity. Immunogenicity may be assessed and modulated using ImmunoPDA™ (see U.S. Ser. Nos. 09/903,378; 10/039,170; 10/339,788; 10/638,995; and 10/754,296all hereby incorporated by reference in their entirety). Modulation, including reduction or enhancement of immunogenicity is accomplished by identifying call II MHC epitopes and modifying them. By “modification” (and its grammatical equivalents) is meant insertion, deletion, substitution or chemical modification of positions identified as part of the epitope. PDA® technology may be used to provide a library of immunogenically modulated CD40L variants. In the instant case, it is preferred that CD40L be assessed and modifications made to reduce immunogenicity.

Expression of CD40L Variants

In a most preferred embodiment, the variant CD40L proteins are expressed in bacterial systems, including but not limited to E. coli. Bacterial expression systems and methods for their use are well known in the art (see Current Protocols in Molecular Biology, Wiley & Sons, and Molecular Cloning—A Laboratory Manual—3^{rd} Ed., Cold Spring Harbor Laboratory Press, New York (2001)). The choice of codons, suitable expression vectors and suitable host cells will vary depending on a number of factors, and may be easily optimized as needed.

Bacterial expression vectors encoding CD40L variants are transformed into bacterial host cells using techniques well known in the art, such as calcium chloride treatment, electroporation, and others.

In a preferred embodiment, bacterial cultures are grown to mid-log phase and expression is induced, for instance with IPTG. Cells are then harvested after 2-24 hours. Protein can be released from the cells using several methods, including sonication, addition of detergents, French press, etc.

In an alternate embodiment, variant CD40L proteins are expressed in non-bacterial systems, including but not limited to yeast, baculovirus, and mammalian expression systems, as well as in vitro expression systems. Suitable protocols are well known in the art [see for example Khandekar et al., Protein Expr Purif. 2001 Nov.; 23(2):301-10 and McGrew et al., Gene. 1997 Mar. 18;187(2):193-200].

Purification of CD40L Variants

In a preferred embodiment, the variant CD40L protein is purified or isolated after expression. Standard purification methods include electrophoretic, molecular, immunological and chromatographic techniques, including ion exchange, hydrophobic, affinity, and reverse-phase HPLC chromatography, and chromatofocusing. For example, the variant CD40L protein may be purified using a standard anti-recombinant protein antibody column. Ultrafiltration and diafiltration techniques, in conjunction with protein concentration, are also useful. For general guidance in suitable purification techniques, see Scopes, R., Protein Purification, Springer-Verlag, NY, 3r ed (1994). The degree of purification necessary will vary depending on the use of the variant CD40L protein. In some instances no purification will be necessary.

Derivatization of CD40L Variants

Once made, the variant CD40L proteins may be covalently or non-covalently modified. Derivatized CD40L variants may exhibit improved solubility, absorption, immunogenicity, pharmacokinetics, and the like. Moieties capable of mediating such effects are disclosed, for example, in Remington's Pharmaceutical Sciences, 16th ed., Mack Publishing Co., Easton, Pa. (1980).

Synthetic Modification

One type of covalent modification includes reacting targeted amino acid residues of a variant CD40L polypeptide with an organic derivatizing agent that is capable of reacting with selected side chains or the N- or C-terminal residues of a variant CD40L polypeptide.

Examples include, but are not limited to, modification to affect binding to human serum albumin, alkylation of any side chain, lipidation, acetylation, acylation, nitrile derivatization of asparagine or glutamine, sulfoxide derivatization of methionine, cysteinyl residues reacted with compounds including alpha-haloacetates, histidyl residues derivatized by reaction with diethylprocarbonate, and lysinyl and reaction of amino terminal residues with compounds such as succinic or other carboxylic acid anhydrides.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of lysine, arginine, and histidine side chains [T. E. Creighton, Proteins: Structure and Molecular Properties, W.H. Freeman & Co., San Francisco, pp. 79-86 (1983)], acetylation of the N-terminal amine, and amidation of any C-terminal carboxyl group.

Derivatization with bifunctional agents is useful, for instance, for cross linking a variant CD40L protein to a water-insoluble support matrix or surface for use in the method for purifying anti-variant CD40L antibodies or screening assays. Commonly used cross linking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio] propioimidate.

Other modifications may be made to the variant CD40L proteins of the present invention, including modifications to the protein that enhance stability, dosage administration (e.g., amphiphilic polymers, see WO 0141812A2, commercially available from Nobex Corporation), clearance (e.g., PEG, aliphatic moieties that affect binding to HSA), and the like.

Glycosylation

The sequence of CD40L variant proteins can be further modified to add or remove glycosylation sites. For example, O-linked glycosylation sites may be altered by adding or removing one or more serine or threonine residues. N-linked glycosylation sites can be altered by incorporating or removing a canonical N-linked glycosylation site, N-X-Y, where N is asparagine, X is any amino acid except for proline and Y is threonine, serine or cysteine. Another means of increasing the number of carbohydrate moieties on the variant CD40L polypeptide is by chemical or enzymatic coupling of glycosides to the polypeptide. Such methods are described in the art, e.g., in WO 87/05330 and in Aplin and Wriston, CRC Crit. Rev. Biochem., pp. 259-306 (1981).

PEGylation

Another type of covalent modification of variant CD40L comprises linking the variant CD40L polypeptide to one of a variety of non-proteinaceous polymers, e.g., polyethylene glycol (“PEG”), polypropylene glycol, or polyoxyalkylenes, in the manner set forth in U.S. Pat. Nos. 4,640,835; 4,496,689; 4,301,144; 4,670,417; 4,791,192 or 4,179,337. These non-proteinaceous polymers may also be used to enhance the ability of a variant CD40L to disrupt receptor binding or to alter the stability, solubility, pharmacokinetics, and/or immunogenicity of the variant CD40L.

In another preferred embodiment, the location of cysteine, lysine, and/or histidine residues in a CD40L variant is modified in order to control the site of PEG attachment. A variety of coupling chemistries may be used to achieve PEGylation, as is well known in the art. Examples, include but are not limited to, the technologies of Shearwater and Enzon. See, Kinstler et al, Advanced Drug Deliveries Reviews, 54, 477-485 (2002) and M J Roberts et al, Advanced Drug Delivery Reviews, 54, 459-476 (2002), both hereby incorporated by reference. In preferred embodiments, PEG-maleimide reagents are used to couple the PEG moiety to cysteine residues on the variant CD40L proteins. These cysteine residues can be one or more of the three naturally occurring cysteines in human CD40L (e.g. C194, C178, C218). Alternatively, one or more of the naturally occurring cysteines can be replaced with alternative amino acids so that more homogeneous coupling is achieved. In further embodiments, novel cysteines can be site-specifically incorporated at judiciously chosen sites within the variant CD40L protein.

Optimal sites for chemical modification can be chosen using a variety of criteria, including but not limited to, visual inspection, structural analysis, sequence analysis and molecular simulation. For example, the fractional accessibility of individual residues can be analyzed to identify modification sites that will not disrupt the monomer structure. In additional preferred embodiments, modification sites are chosen such that the distance from the modification site to another CD40L monomer is maximal. In additional preferred embodiments, rational PEGylation methods may be used to determine optimal PEG attachment sites (see for example, U.S. Ser. No. 60/459,094, filed Mar. 31, 2003, hereby incorporated by reference in its entirety). In additional embodiments, it is possible that receptor binding disruption may occur and may be beneficial to the activity of the CD40L variants of this invention.

In another aspect of covalent modification, addition of PEG or other moieties may prevent exchange by blocking the accessibility of variants to the membrane-associated ligand. Introducing modifications such as PEG molecules creates steric hindrance to such interactions. In this way, variants can be constructed that are specific to the soluble form of the ligand. The introduction of the PEG at some sites may abrogate the ability of the molecule to inhibit transmembrane CD40L. For example, human transmembrane TNF-alpha is inhibited by non-PEGylated TNF-alpha variants, but is not inhibited by at least one PEGylated TNF-alpha variant.

Variant CD40L proteins of the invention that can specifically antagonize endogenous soluble CD40L may have increase safety and reduced side effects. Elevated levels of soluble CD40L have been established for a variety of disease conditions, including but not limited to: chronic renal failure, diabetes, inflammatory bowel disease, autoimmune thrombocytopenic purpura, Hodgkin's disease, rheumatoid vasculitis, systemic lupus erythrematosis, chronic lymphocytic leukaemia, preeclampsia, sickle cell anemia, hypercholesterolemia, atherosclerosis, and numerous cardiovascular conditions. Variant CD40L proteins that specifically inhibit soluble CD40L may find therapeutic use in one or more of these disease conditions.

Variant CD40L proteins of the invention that can antagonize both membrane and soluble forms of CD40L, including DN-CD40L and ciCD40L variants, may find therapeutic use in any number of disease conditions for which antagonism of immune cell activation is desirable, including a variety of inflammatory and autoimmune diseases. Such diseases include, but are not limited to: systemic lupus erythematosus (SLE), rheumatoid and collagen-induced arthritis, pre- and post-transplantation immunosuppression, psoriasis, multiple sclerosis, allergic encephalitis, acute and chronic graft versus host disease, Crohn's disease, diabetes, Hodgkin's and non-Hodgkin's Lymphomas (NHL), chronic renal failure, mixed connective tissue disease, sickle cell anemia, inflammatory bowel disease, Hodgkin's disease, rheumatoid vasculitis, chronic lymphocytic leukaemia, preeclampsia and cardiovascular conditions including atherosclerosis, thrombocytopenia (Purpura).

In additional embodiments, variant CD40L proteins of the invention that can antagonize platelet activation may find use in a variety of cardiovascular and related diseases.

Circular Permutation and Cyclization

In another preferred embodiment, the wild-type CD40L or variants generated by the invention may be circularly permuted. All natural proteins have an amino acid sequence beginning with an N-terminus and ending with a C-terminus. The N- and C-termini may be joined to create a cyclized or circularly permutated CD40L proteins while retaining or improving biological properties (e.g., such as enhanced stability and activity) as compared to the wild-type protein. In the case of a CD40L protein, a novel set of N- and C-termini are created at amino acid positions normally internal to the protein's primary structure, and the original N- and C-termini are joined via a peptide linker consisting of from 0 to 30 amino acids in length (in some cases, some of the amino acids located near the original termini are removed to accommodate the linker design). In a preferred embodiment, the novel N- and C-termini are located in a non-regular secondary structural element, such as a loop or turn, such that the stability and activity of the novel protein are similar to those of the original protein. In a further preferred embodiment PDAe technology may be used to further optimize the CD40L variant, particularly in the regions created by circular permutation. These include the novel N- and C-termini, as well as the original termini and linker peptide. In addition, a completely cyclic CD40L may be generated, wherein the protein contains no termini. This is accomplished utilizing intein technology. Thus, peptides can be cyclized and in particular inteins may be utilized to accomplish the cyclization.

Various techniques may be used to permutate proteins. See U.S. Pat. No. 5,981,200; Maki K, Iwakura M., Seikagaku. 2001 January; 73(1): 42-6; Pan T., Methods Enzymol. 2000; 317:313-30; Heinemann U, Hahn M., Prog Biophys Mol. Biol. 1995; 64(2-3): 121-43; Harris M E, Pace N R, Mol Biol Rep. 1995-96; 22(2-3): 115-23; Pan T, Uhlenbeck O C., 1993 Mar. 30; 125(2): 111-4; Nardulli A M, Shapiro D J. 1993 Winter; 3(4): 247-55, EP 1098257 A2; WO 02/22149; WO 01/51629; WO 99/51632; Hennecke, et al., 1999, J. Mol. Biol., 286, 1197-1215; Goldenberg et al J. Mol. Biol. 165, 407-413 (1983); Luger et al, Science, 243, 206-210 (1989); and Zhang et al., Protein Sci 5, 1290-1300 (1996); all hereby incorporated by reference.

Linkers

In a preferred embodiment, a linker peptide is chosen such that the two CD40L variant monomers assume a conformation that allows binding to the CD40L receptors. The linker peptide should have a length that is adequate to link two CD40L variant monomers in such a way that they assume the correct conformation relative to one another so that they retain the desired activity as antagonists of the native CD40L protein. Suitable lengths for this purpose include at least one and not more than 30 amino acid residues. Preferably, the linker is from about 1 to 30 amino acids in length, with linkers of 1, 2, 3, 4, 5, 6, 7, 8, 9, 10, 11, 12, 13, 14, 15, 16, 17, 18 19 and 20 amino acids in length being preferred. See also WO 01/25277, incorporated herein by reference in its entirety.

In addition, the amino acid residues selected for inclusion in the linker peptide should exhibit properties that do not interfere significantly with the activity of the polypeptide. Thus, the linker peptide on the whole should not exhibit a charge that would be inconsistent with the activity of the polypeptide, or interfere with internal folding, or form bonds or other interactions with amino acid residues in one or more of the monomers that would seriously impede the binding of receptor monomer domains.

Useful linkers include glycine-serine polymers (including, for example, (GS)n, (GSGGS)n (GGGGS)n and (GGGS)n, where n is an integer of at least one), glycine-alanine polymers, alanine-serine polymers, and other flexible linkers such as the tether for the shaker potassium channel, and a large variety of other flexible linkers, as will be appreciated by those in the art. Glycine-serine polymers are preferred since both of these amino acids are relatively unstructured, and therefore may be able to serve as a neutral tether between components. Secondly, serine is hydrophilic and therefore able to solubilize what could be a globular glycine chain. Third, similar chains have been shown to be effective in joining subunits of recombinant proteins such as single chain antibodies.

Suitable linkers may also be identified by screening databases of known three-dimensional structures for naturally occurring motifs that can bridge the gap between two polypeptide chains. Another way of obtaining a suitable linker is by optimizing a simple linker, e.g., (Gly4Ser)n, through random mutagenesis. Alternatively, once a suitable polypeptide linker is defined, additional linker polypeptides can be created by application of PDA™ technology to select amino acids that more optimally interact with the domains being linked. Other types of linkers that may be used in the present invention include artificial polypeptide linkers and inteins. In another preferred embodiment, disulfide bonds are designed to link the two receptor monomers at inter-monomer contact sites. In one aspect of this embodiment the two receptors are linked at distances <5 Angstroms. In addition, the variant CD40L polypeptides of the invention may be further fused to other proteins, if desired, for example to increase expression or stabilize the protein.

Single Chain Dominant-Negative Polypeptides

Multiple strategies for covalent linkage of monomers exist. These included, but are not limited to: polypeptide linkages between N and C-termini of two domains, made up of zero or more amino acids (resulting in single chain polypeptides comprising multiple domains); linkage via a disulfide bond between monomers; linkage via chemical cross-linking agents.

Multiple strategies exist for modification of individual domains such that receptor binding is removed (or reduced). These include, but are not limited to: amino acid modifications that create steric repulsion between ligand domain and receptor; modifications that create electrostatic repulsion; modifications that create unfavorable desolvation of amino acids; and chemical modification of amino acids at the ligand/receptor interface (e.g. PEGylation or glycosylation).

Fusion Constructs

Variant CD40L polypeptides of the present invention may also be fused to another, heterologous polypeptide or amino acid sequence to form a chimera. The chimeric molecule may comprise a fusion of a variant CD40L polypeptide with an immunoglobulin or a particular region of an immunoglobulin such as the Fc or Fab regions of an IgG molecule.). In some embodiments, for example in the creation of animal models of disease, fusion proteins comprising the variant CD40L proteins with other sequences may be done, for example using fusion partners comprising labels (e.g. autofluorescent proteins, survival and/or selection proteins), stability and/or purification sequences, toxins, variant proteins from other members of the superfamily (e.g. analogous to the creation of “bi-specific antibodies”) or any other protein sequences of use. Additional fusion partners are described below. In some instances, the fusion partner is not a protein.

In another embodiment, the CD40L variant is fused with human serum albumin to improve pharmacokinetics. In another preferred embodiment, the CD40L variant is conjugated to an antibody, preferably an anti-variant CD40L protein antibody.

In a further embodiment, CD40L is fused to a cytotoxic agent. In this method, the CD40L fusion acts to target the cytotoxic agent to tumor tissue or cells, resulting in a reduction in the number of afflicted cells. Such an approach thereby reduces symptoms associated with cancer and CD40L protein related disorders. Cytotoxic agents include, but are not limited to, diphtheria A chain, exotoxin A chain, ricin A chain, abrin A chain, curcin, crotin, phenomycin, enomycin and the like, as well as radiochemicals.

In another embodiment, variant CD40L proteins of the invention are fused to trimerizing domains, which include but are not limited to isoleucine-zipper motifs (as disclosed in U.S. Pat. No. 6,264,951, hereby incorporated by reference in its entirety).

Peptide Tags

Various tag polypeptides and their respective antibodies are well known in the art. Epitope tags may be placed at the amino- or carboxyl-terminus of the variant CD40L proteins to enable antibody detection. Also, the epitope tag enables the variant CD40L protein to be readily purified by affinity purification. Examples of peptide tags include, but are not limited to, poly-histidine (poly-His) or poly-histidine-glycine (poly-His-Gly) tags; the flu HA tag polypeptide [Field et al., Mol. Cell. Biol. 8:2159-2165 (1988)]; the c-myc tag [Evan et al., Molecular and Cellular Biology, 5:3610-3616 (1985)]; the Herpes Simplex virus glycoprotein D (gD) tag [Paborsky et al., Protein Engineering, 3(6):547-553 (1990)], the Flag-peptide [Hopp et al., BioTechnology 6:1204-1210 (1988)]; the KT3 epitope peptide [Martin et al., Science 255:192-194 (1992)]; tubulin epitope peptide [Skinner et al., J. Biol. Chem. 266:15163-15166 (1991)]; and the T7 gene 10 protein peptide tag [Lutz-Freyermuth et al., Proc. Natl. Acad. Sci. U.S.A. 87:6393-6397 (1990)].

Labels

In one embodiment, the variant CD40L protein is modified by the addition of one or more labels. For example, labels that may be used are well known in the art and include but are not limited to biotin, tag and fluorescent labels (e.g. fluorescein). These labels may be used in various assays as are also well known in the art to achieve characterization.

Assays

Variant CD40L proteins may be experimentally tested and validated using in vivo and in vitro assays. Suitable assays include, but are not limited to, activity assays and binding assays. For example, CD40L activity assays, such as B-cell proliferation assays (Spriggs et. al., JEM 176, 1543-1550 (1992)), the NF-κB pathway activation assays (Wei et al., Endocrinology 142, 1290-1295, (2001)), c-Jun (Srivastava et al., JBC 276, 8836-8840 (2001)) transcription factor activation assays, or B cell surface receptor activation-induced apoptosis rescue assay (Gauld et. al., J. Immunol. 168, 3855-3864 (2002)) for monitoring signaling through CD40 are screens that may be utilized in identifying CD40L variants that are antagonists of wild-type CD40L.

In a preferred embodiment, binding affinities for the following interactions are determined and compared: 1) variant CD40L oligomer formation, 2) wild-type CD40L oligomer formation, 3) variant CD40L binding to CD40, 4) wild-type CD40L binding to CD40, 5) variant CD40L binding to alpha IIb-beta3 integrin, 6) wild-type CD40L binding to alpha IIb-beta3 integrin, 7) CD40L variant binding to CD40, and 8) CD40L variant binding to alpha IIb-beta3 integrin. Suitable assays include, but are not limited to, quantitative comparisons comparing kinetic and equilibrium binding constants. The kinetic association rate (kₒₙ) and dissociation rate (k_{off}), and the equilibrium binding constants (K_{d}) may be determined using surface plasmon resonance on a BIAcore® instrument following the standard procedure in the literature [Pearce et al., Biochemistry 38:81-89 (1999)]. Several alternative methods can also be used to determine binding affinity and kinetics.

In an alternate embodiment, binding affinities for the following interactions are determined and compared: 1) variant CD40L oligomer formation, 2) wild-type CD40L oligomer formation, 3) variant CD40L binding to CD40, 4) wild-type CD40L binding to CD40, 5) variant CD40L binding to alpha IIb-beta3 integrin, 6) wild-type CD40L binding to alpha IIb-beta3 integrin, 7) CD40L variant binding to CD40, and 8) CD40L variant binding to alpha IIb-beta3 integrin. In a preferred embodiment, CD40L variant proteins do not bind alpha IIb-beta3 integrin. Alpha IIb-beta3 integrin is a decoy receptor and functions by binding and sequestering CD40L. The CD40L variant antagonists, agonists, and competitive inhibitors that do not bind alpha IIb-beta3 integrin will be more effective therapeutics as they will not be sequestered by alpha IIb-beta3 integrin and they will not activate endogenous alpha IIb-beta3 integrin.

CD40L Binding Assays

AlphaScreen™ Binding Assays

The AlphaScreen™ (Bosse R., Illy C., and Chelsky D (2002). Principles of AlphaScreen™ PerkinElmer Literaure Aplication Note Ref# s4069 (lifesciences.perkinelmer.com/Notes/S4069-0802.pdf) is one approach for determining the relative binding of a ligand to a receptor. AlphaScreen™ is a bead-based non-radioactive luminescent proximity assay where the donor beads are excited by a laser at 680 nm to release singlet oxygen. The singlet oxygen diffuses and reacts with the thioxene derivative on the surface of acceptor beads leading to fluorescence emission at ˜600 nm. The fluorescence emission occurs only when the donor and acceptor beads are brought into close proximity by molecular interactions occurring when each is linked to ligand and receptor respectively. This ligand-receptor interaction can be competed away using receptor-binding variants while non-binding variants will not compete. Therefore, variant CD40L proteins can be assessed for CD40 and alpha IIb-beta3 integrin binding using the AlphaScreen™ approach.

CD40L variants can also be tested to determine whether they are capable of forming mixed oligomers including but not limited to mixed trimers. In a preferred embodiment, this is accomplished by labeling wild-type CD40L and variant CD40L with distinguishable tags, combining wild-type and variant CD40L, and screening for oligomers that contain both tag types. For example, FLAG-tagged wild-type CD40L and His-tagged variant CD40L can be combined, and sandwich ELISAs can be performed to identify trimers that contain both FLAG and His tag. Another alternative is to run native gels and perform Western blots using both anti-FLAG and anti-His tag antibodies. This method relies on the fact that FLAG and His tags significantly perturb protein migration in native gels. As will be appreciated by those in the art, many alternate protocols could also be used to measure the formation of mixed trimers.

Therapeutic Application of CD40L Variants

Once made, the variant CD40L proteins and nucleic acids of the invention find use in a number of applications. In a preferred embodiment, the variant CD40L proteins are administered to a patient to treat a CD40L related disorders. Table 1, supra, shows that there are many possible indications that are affected by CD40L-CD40 interaction. Indications include but are not limited to cardiovascular (e.g. atherosclerosis, thrombocytopenia, etc.), pre- and post-transplant immunosuppression, systemic lupus erythromus (SLE), oncological or anti-tumor, multiple sclerosis, autoimmune related conditions such as psoriasis, rheumatoid arthritis, Crohn's disease, Graft versus Host Disease (GVHD), allergic encephalitis, chronic renal failure, sickle cell anemia, mixed connective tissue disease, inflammatory bowel disease, Hodgkin's disease, rheumatoid vasculitis, chronic lymphocytic leukaemia, preeclampsia, etc.

Pharmaceutical Composition

The pharmaceutical compositions of the present invention comprise a variant CD40L protein in a form suitable for administration to a patient. In the preferred embodiment, the pharmaceutical compositions are in a water-soluble form, for example as pharmaceutically acceptable salts. Particularly preferred are the ammonium, potassium, sodium, calcium, and magnesium salts.

The pharmaceutical compositions may also include one or more of the following: carrier proteins such as serum albumin; buffers such as NaOAc; fillers such as microcrystalline cellulose, lactose, corn and other starches; binding agents; sweeteners and other flavoring agents; coloring agents; and polyethylene glycol. Additives are well known in the art, and are used in a variety of formulations.

In a further embodiment, the variant CD40L proteins are added in a micellular formulation; see U.S. Pat. No. 5,833,948, hereby expressly incorporated by reference in its entirety.

Combinations of pharmaceutical compositions may be administered. Moreover, the compositions may be administered in combination with other therapeutics.

Depending upon the manner of introduction, the pharmaceutical composition may be formulated in a variety of ways. The concentration of the therapeutically active variant CD40L protein in the formulation may vary from about 0.1 to 100 weight %. In another preferred embodiment, the concentration of the variant CD40L protein is in the range of 0.003 to 1.0 molar, with dosages from 0.03, 0.05, 0.1, 0.2, and 0.3 millimoles per kilogram of body weight being preferred.

Also, sustained release or controlled release formulations may be used for the compositions of the present invention. For example, ProLease® (commercially available from Alkermes) a microsphere-based delivery system composed of the desired bioactive molecule incorporated into a matrix of poly-DL-lactide-co-glycolide (PLG) and other pharmaceutically compatible polymeric matrices may be used to create sustained release formulations.

Route of Administration

The administration of the variant CD40L proteins of the present invention, preferably in the form of a sterile aqueous solution, may be done in a variety of ways, including, but not limited to, orally, subcutaneously, intravenously, intranasally, intraotically, transdermally, topically (e.g., gels, salves, lotions, creams, etc.), intraperitoneally, intramuscularly, intrapulmonary (e.g., AERx® inhalable technology commercially available from Aradigm or Inhance™ pulmonary delivery system commercially available from Inhale Therapeutics), vaginally, rectally, embedded in bones, joints, prosthesis, or collagen sponges, or intraocularly. In some instances, for example, in the treatment of wounds, inflammation, etc., the variant CD40L protein may be directly applied as a solution, salve, cream or spray. The TNF-alpha molecules of the present may also be delivered by bacterial or fungal expression into the human system (e.g., WO 04046346 A2, hereby incorporated by reference). Depending upon the manner of introduction, the pharmaceutical composition may be formulated in a variety of ways.

Dosing

In a preferred embodiment, a therapeutically effective dose of a variant CD40L protein is administered to a patient in need of treatment. The exact dose will depend on the purpose of the treatment, and will be ascertainable by one skilled in the art using known techniques. As is known in the art, adjustments for variant CD40L protein degradation, systemic versus localized delivery, and the rate of new protease synthesis, as well as the age, body weight, general health, sex, diet, time of administration, drug interaction and the severity of the condition may be necessary, and will be ascertainable with routine experimentation by those skilled in the art.

Gene Therapy

Similarly, nucleic acid encoding the variant CD40L proteins (including both the full-length sequence, partial sequences, or regulatory sequences of the variant CD40L coding regions) may be administered in gene therapy applications.

In a preferred embodiment, the nucleic acid encoding the variant CD40L proteins (including both the full-length sequence, partial sequences, or regulatory sequences of the variant CD40L coding regions) may also be used in gene therapy. In gene therapy applications, genes are introduced into cells in order to achieve in vivo synthesis of a therapeutically effective genetic product, for example for replacement of a defective gene. Antisense RNA and DNA can be used as therapeutic agents for blocking the expression of certain genes in vivo. It has already been shown that short antisense oligonucleotides can be imported into cells where they act as inhibitors (see Zamecnik et al., Proc. Natl. Acad. Sci. U.S.A. 83:4143-4146 (1986)). The oligonucleotides can be modified to enhance their uptake, e.g. by substituting their negatively charged phosphodiester groups by uncharged groups.

There are a variety of techniques available for introducing nucleic acids into viable cells. The currently preferred in vivo gene transfer techniques include transfection with viral (typically retroviral) vectors and viral coat protein-liposome mediated transfection [Dzau et al., Trends in Biotechnology 11:205-210 (1993)]. In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262:4429-4432 (1987); and Wagner et al., Proc. Natl. Acad. Sci. U.S.A. 87:3410-3414 (1990). For review of gene marking and gene therapy protocols see Anderson et al., Science 256:808-813 (1992).

In a preferred embodiment, variant CD40L genes are administered as DNA vaccines, either single genes or combinations of variant CD40L genes. Naked DNA vaccines are generally known in the art. Brower, Nature Biotechnology, 16:1304-1305 (1998). Methods for the use of genes as DNA vaccines are well known to one of ordinary skill in the art, and include placing a variant CD40L gene or portion of a variant CD40L gene under the control of a promoter for expression in a patient in need of treatment.

In a preferred embodiment, the DNA vaccines include a gene encoding an adjuvant molecule with the DNA vaccine. Such adjuvant molecules include cytokines that increase the immunogenic response to the variant CD40L polypeptide encoded by the DNA vaccine. Additional or alternative adjuvants are known to those of ordinary skill in the art and find use in the invention.

References all of which are incorporated by reference in their entirety.
Tan et al, Curr. Op. Pharmacology, 2:445-451 (2002)
Van Kooten et al, Curr. Op. Immunology 9:330-337 (1997)
Propst et al, J Biol Chem, 277 (40) 37054-37063 (2002)
Grewal, et al, Annu. Rev. Immunol, 16: 111-35 (1998)
Lutgens et al, TCM, 12(1) 27 (2002)
Kweon et al, J. Clin. Investigations, 109(2): 171-173 (2002)
Qian et al, PNAS, 99(14): 9386-9391 (2002)
Crow et al, Curr. Op. Rheumatology, 13:361-369 (2001)
Schwabe et al, American Association of Immunologists, p 1767, (2001)
Yin et al, Transplantation, 74(3) 345-354 (2002)
Bavendiek et al, J. Biol. Chem., 277 (28): 25032-25039 (2002)
Andre' et al, Circulation, 106: 896-899 (2002)
Morris et al, J. Biol. Chem., 274(1): 418-422 (1999)
Schoenbeck et al, PNAS, 97(13): 7458-7463 (2002)
Peitravalle et al, J. Biol. Chem, 271(11): 5965-5967 (1996)
Lee et al, Proc. Natl. Acad. Sci USA, 96: 1421-1426 (1999)
Lee et al., Blood. Eprint, 9 Sep. (2004)
Faucher and Pliska, Eur. J. Med. Chem., 18, 369-375 (1983)
Mann et al, J. Biol. Chem., 277(19): 17125-17135 (2002)
Haenninen et al, J. Clin. Invest., 109: 261-267 (2002)
Tamada et al, J. Clin. Invest., 109: 549-557 (2002)
Town et al., Neurochemistry International, 39: 371-380 (2001)
Graf, et al., Eur. J. Immunol. 22 (12), 3191-3194 (1992)
Spriggs, et al, J. Exp. Med. 176 (6), 1543-1550 (1992)
Hollenbaugh, D. et al, EMBO J. 11 (12), 4313-4321 (1992)
Gauchat, J. F., et al, FEBS Lett. 315 (3), 259-266 (1993)
Prasad, et al, PNAS, 100(21): 12367-12371 (2003)
WO 96/40918 A2; WO 98/01145 A1; WO 99/27948 A2; WO 01/96397 A2; WO 02/089730 A2; WO 95/14487 A1
EP 00585943 B1

### EXAMPLE 1

Measuring Exchange Between CD40L Trimers

In order to measure the kinetics of exchange between CD40L trimers in solution we developed a novel spectroscopic assay. This technique utilizes the polarization anisotropy differences between homotrimers of fluorescently modified CD40L and heterotrimers formed between fluorescent and unlabeled CD40L molecules. Since this assay is carried out in a real-time sampling device, we can measure the formation of CD40L heterotrimers as a function of time. Furthermore, this assay is sensitive to a variety of buffers and/or excipients thereby enabling a detailed kinetic analysis of CD40L exchange in solution.

This assay necessitates a fluorescently labeled CD40L trimer that at limiting concentrations may be used as a tracer to monitor exchange. We generate a CD40L variant and specifically label it with Alexa568 maleimide. Addition of surfactant excipients catalyzes the exchange between CD40L homotrimers. We mix together 1 ug/mL Alexa568 CD40L alone or with a PEGylated sample (e.g. 100 ug/mL PEG-5k A145R/197T) CD40L variant in a 96-well assay format and began anisotropy measurements. After ˜2000 seconds the instrument is paused and 0.5% polysorbate-20 or polysorbate-80 is added and the measurement is resumed.

Next we characterize the spectral properties of this modified CD40L and demonstrate that we can use it to measure exchange between Alexa568 and unlabeled CD40Ls. Polarization anisotropy demonstrates heterotrimer formation between CD40L homotrimers. We mix 1 ug/mL Alexa568 CD40L either alone or with 0.1 ug/mL, 1 ug/mL, 3 ug/mL, 5 ug/mL, 7 ug/mL, 10 ug/mL, or 50 ug/mL PEG-5k variant CD40L (or non PEGylated CD40L variant) in 96-well assay format. These reactions are supplemented with 0.02% igepal and the plate is immediately placed into the instrument to begin anisotropy measurements. Once the time-course is completed the end-point samples are analyzed using native PAGE to determine the extent of Alexa568 CD40L sequestration into heterotrimers. We anticipate observing a strong correlation between increasing anisotropy values and the sequestration of Alexa568 CD40L from the quickly migrating homotrimer at the bottom of the gel to the slowly moving heterotrimer at the top of the gel.

Native PAGE analysis demonstrates that anisotropy changes correlate with the decreased mobility of CD40L heterotrimers on gels. Furthermore this assay has further utility because it is compatible with both modified (i.e. PEGylated) and unmodified cold CD40Ls, and it is highly specific for exchange between TNFSFs (i.e. CD40L fails to exchange with RANKL). Exchange in the absence of excipients reveals CD40L variants with improved exchange kinetics. We use polarization anisotropy to measure the heterotrimer formation as the reaction relaxed to equilibrium. Our results should suggest that CD40L variants containing the specific mutations exchange faster than native CD40L. Finally, other methods require either solid-phase (i.e. sandwich ELISA or RIA), or acrylamide gels (i.e. native PAGE analysis or IEF) to resolve the end products of heterotrimer formation. This assay is superior to currently utilized methods because it allows kinetic analysis in solution.

This assay provides unexpected results in that we can measure a change in polarization anisotropy without any apparent change in molecular weight (i.e. exchange between Alexa568 CD40L and cold unPEGylated CD40L). Our experimental analysis will suggest that there is an appreciable amount of fluorescent enhancement observed upon heterotrimer formation. We will exploite this change with the aid of polarizing filters to increase sensitivity and generate the anisotropy differences shown in the above examples.

This exchange protocol enables the development of additional criteria useful in evaluating DN-CD40L variants. This assay is implemented, under excipient-free conditions to evaluate large numbers of CD40L variants for their exchange properties. Using this data we were able to determine that several DN variants as well as double and triple mutants including at least one of these mutations) having improved exchange properties even in the absence of excipients.

Whereas particular embodiments of the invention have been described above for purposes of illustration, it will be appreciated by those skilled in the art that numerous variations of the details may be made without departing from the invention as described in the appended claims. All references cited herein are expressly incorporated by reference.

### EXAMPLE 2

Optimized PEGylation of CD40L

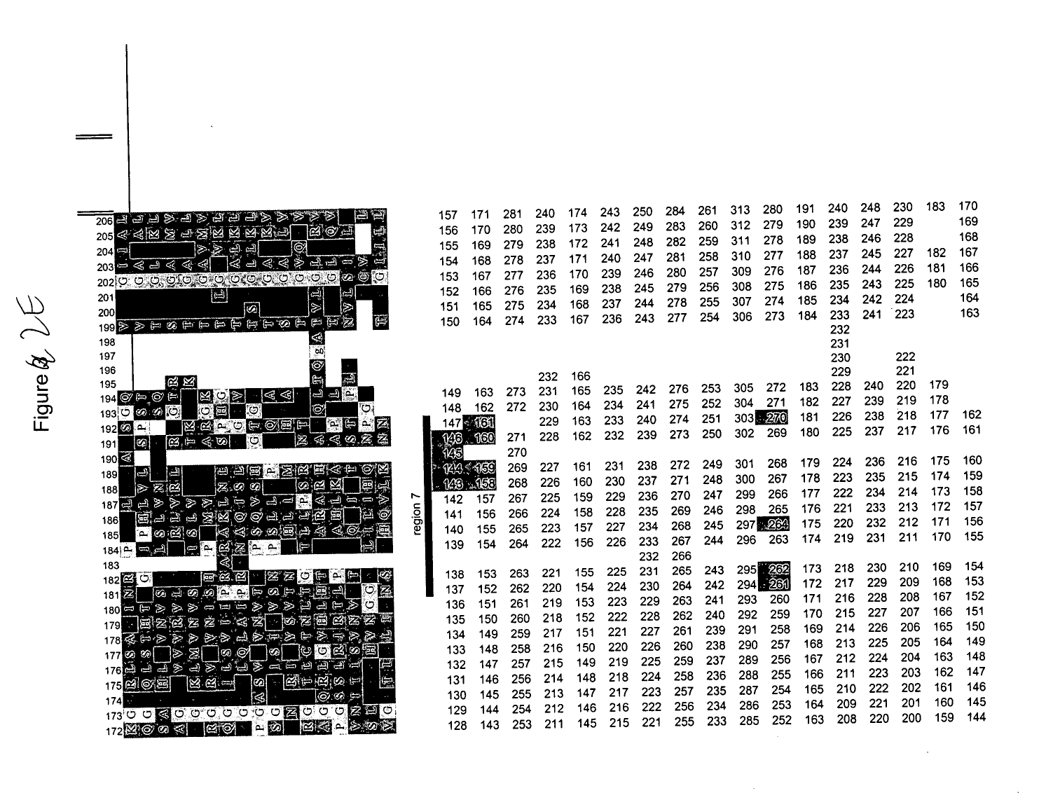

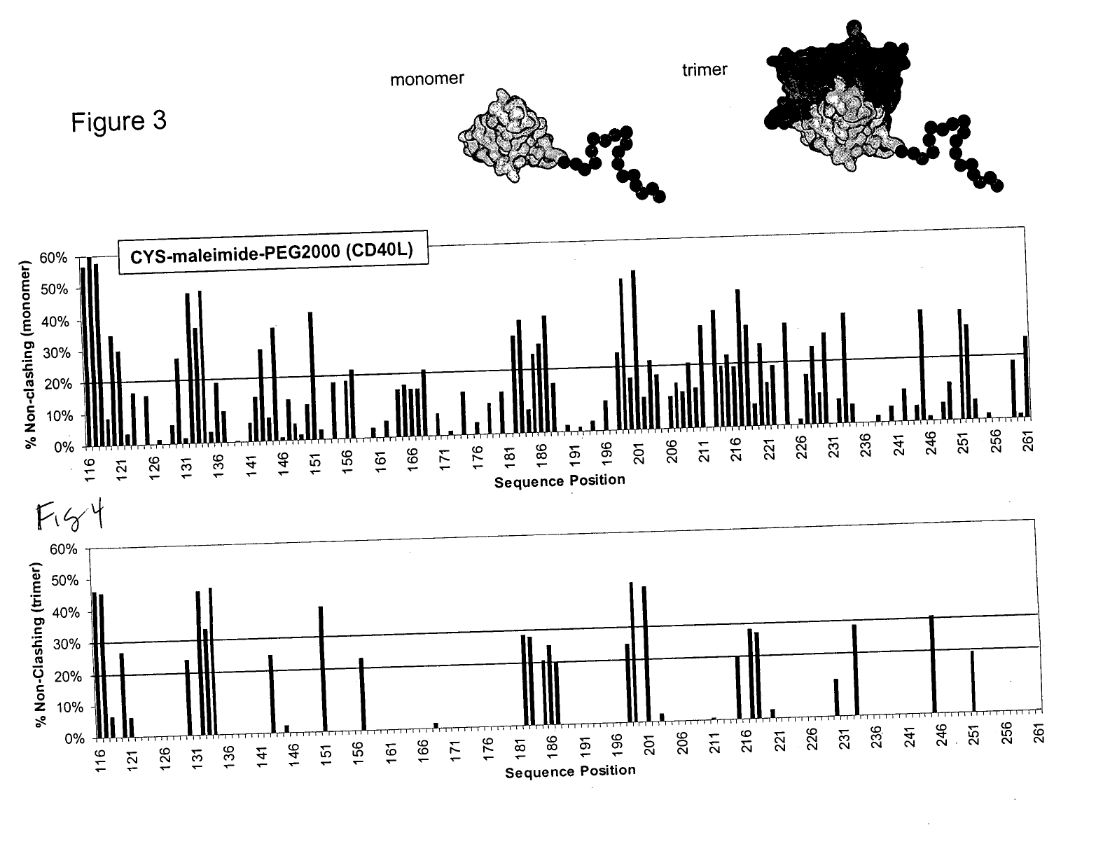

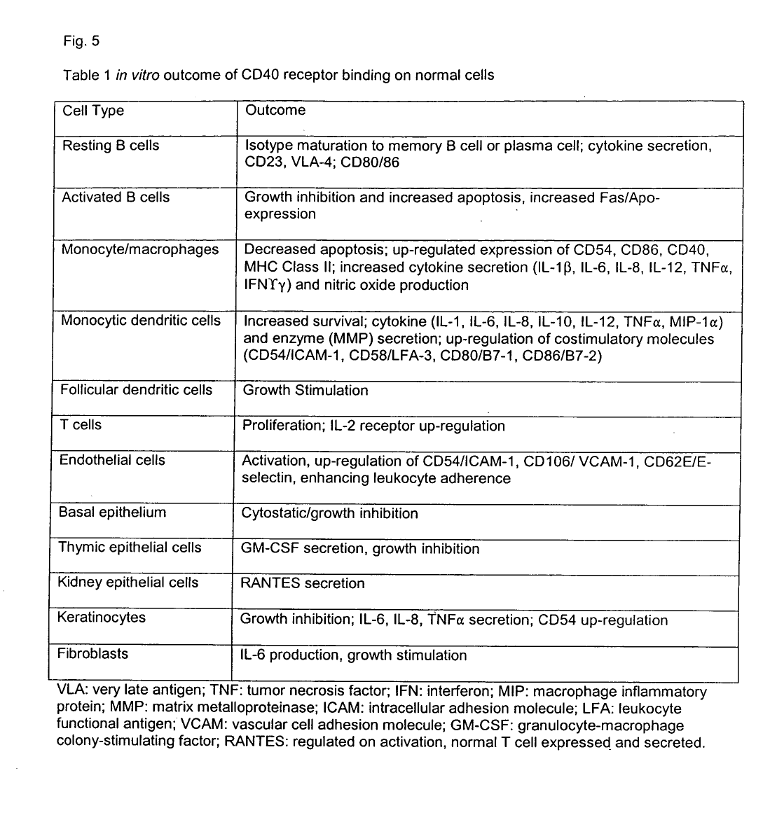

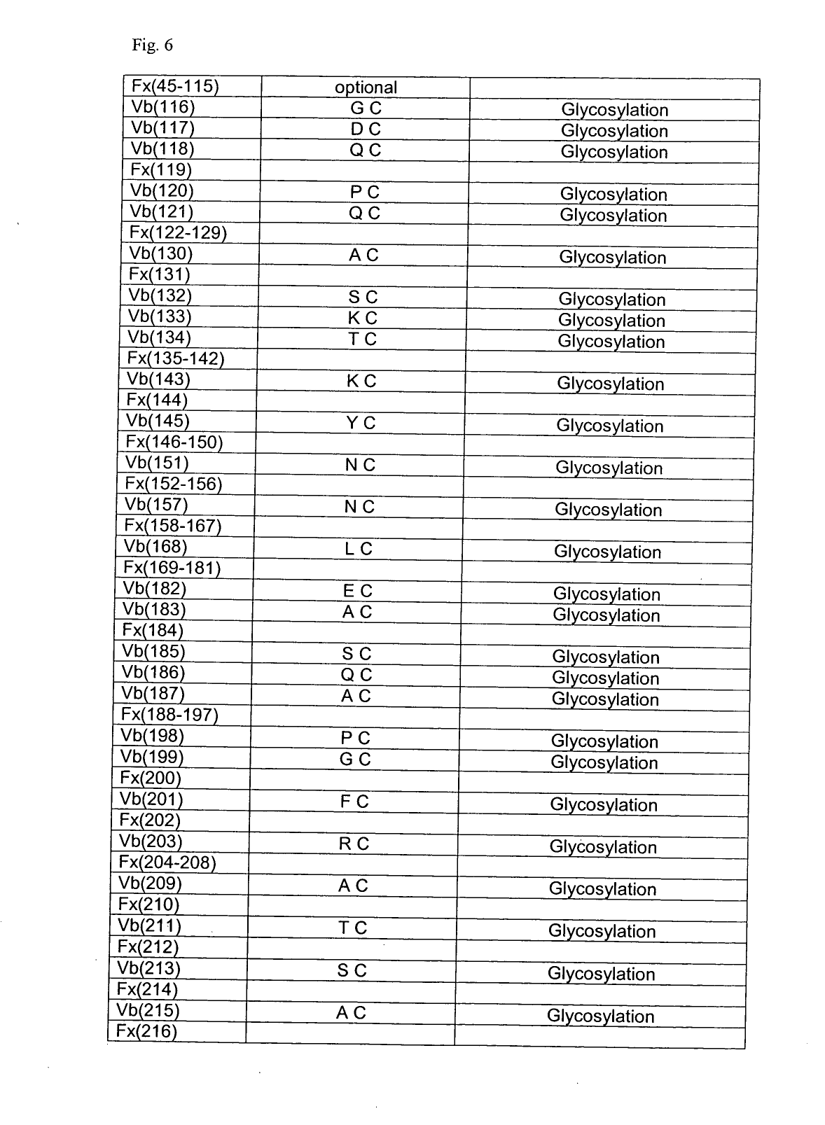

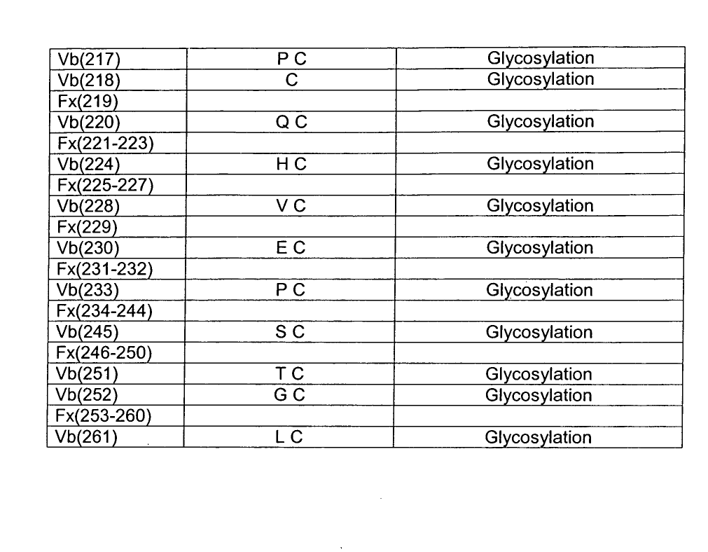

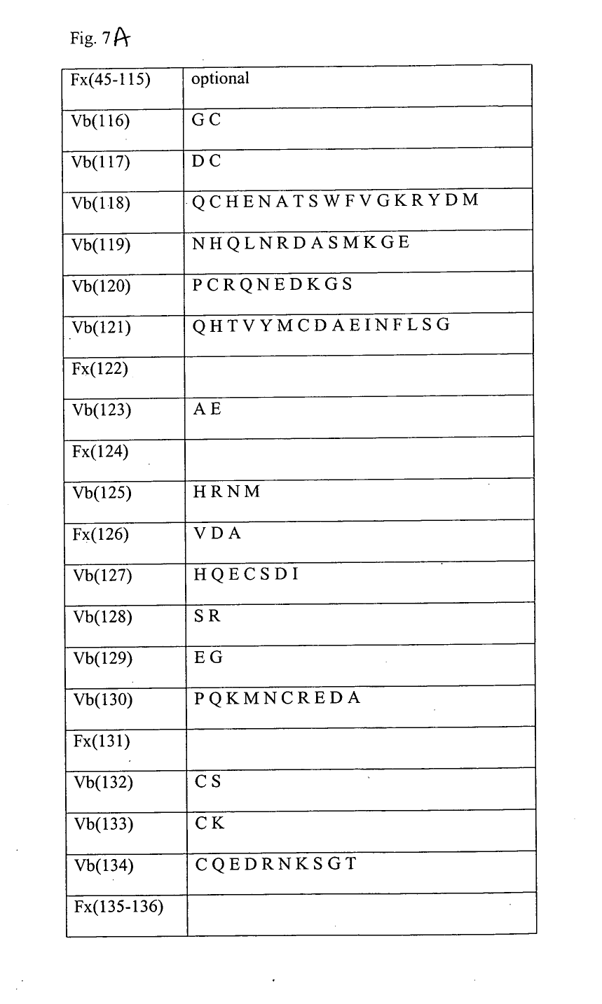

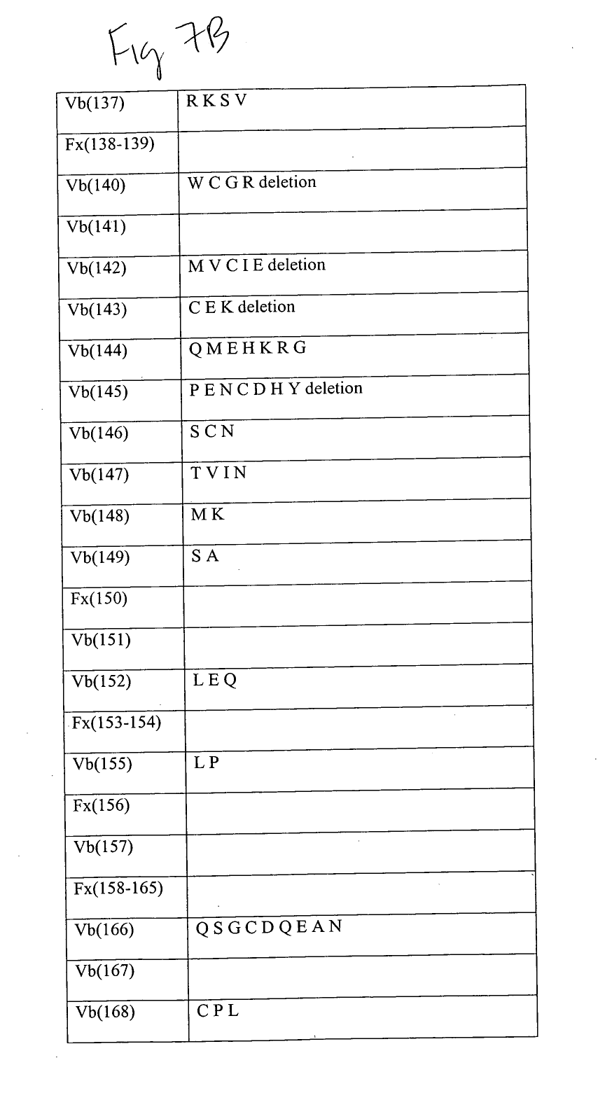

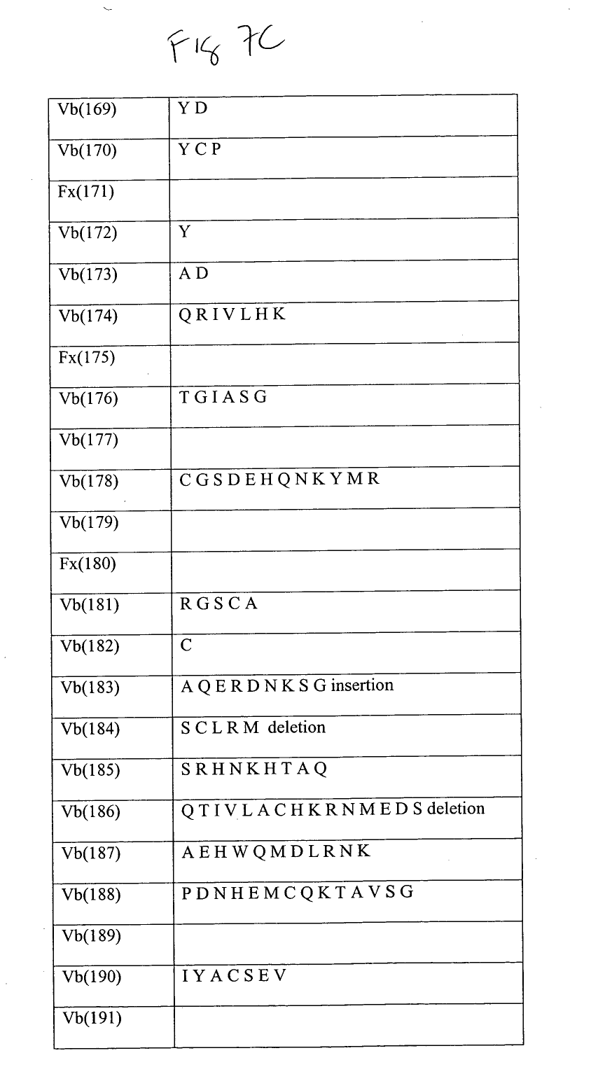

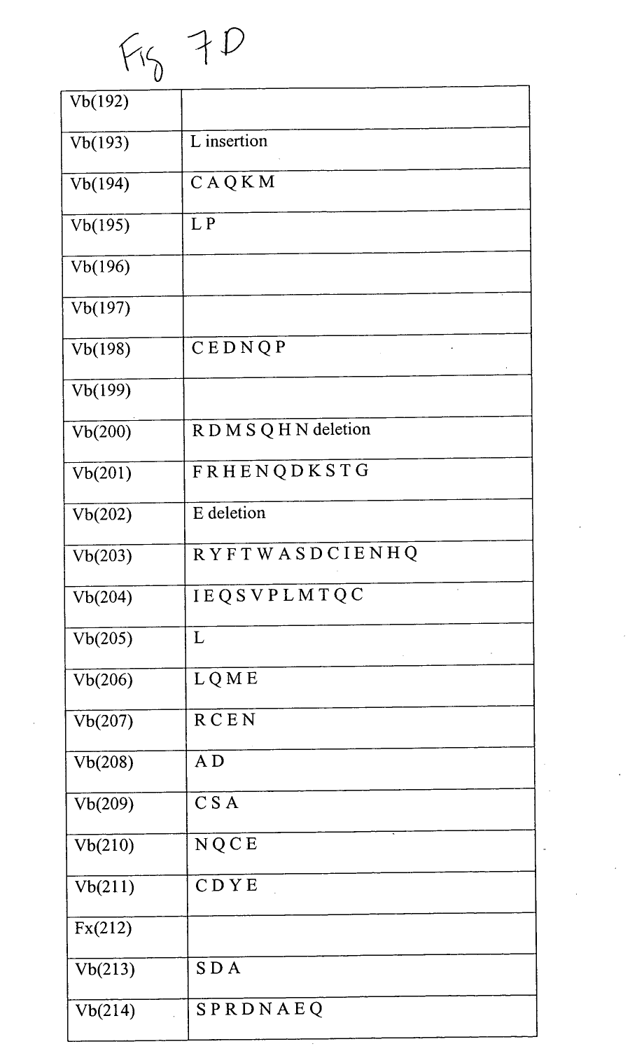

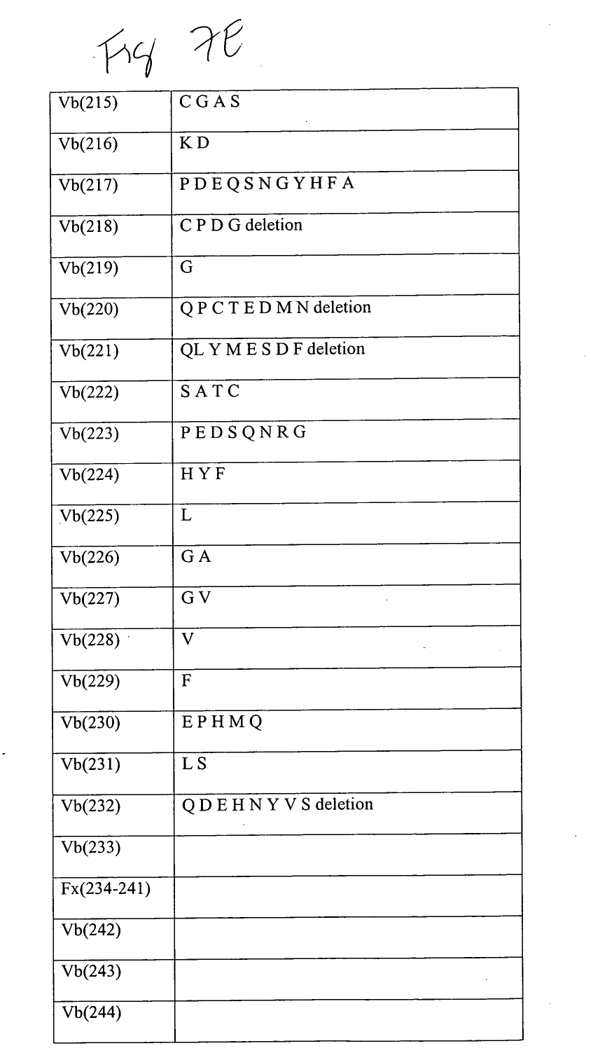

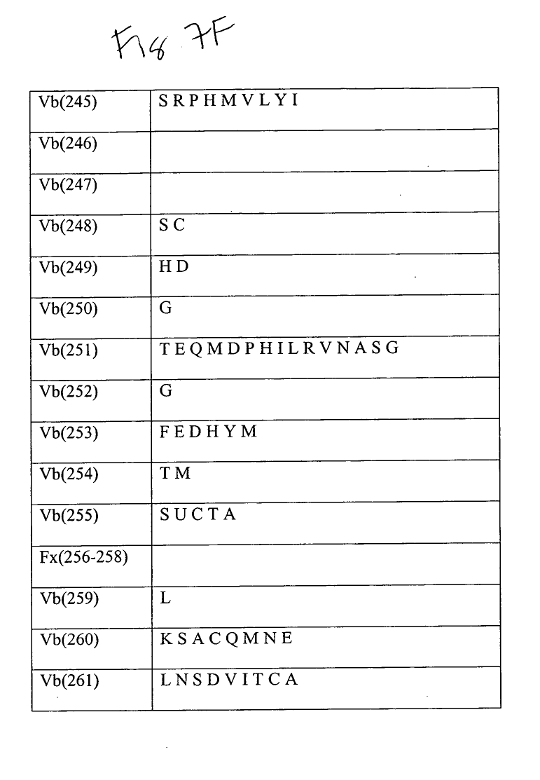

As with most therapeutic proteins, the PEGylation of CD40L is expected to improve its pharmacokinetic properties in a patient. The methods of the present invention have been used to select optimal PEGylation sites in CD40L (see FIG. 3) based on Protein Data Bank structure 1ALY. The simulation data was first analyzed to identify sites with high coupling efficiency. For PEG2000, sites for which greater than 20% of the simulated PEG chains are non-clashing in the free state are considered optimal sites for attachment (see FIG. 3, top chart). These sites include Gly116, Asp117, Gln118, Pro120, Gln121, Ala130, Ser132, Lys133, Thr134, Lys143, Tyr145, Asn151, Asn157, Leu168, Glu182, Ala183, Ser185, Gln186, Ala187, Pro198, Gly199, Phe201, Arg203, Ala209, Thr211, Ser213, Ala215, Pro217, Cyd218, Gln220, His224, Val228, Glu230, Pro233, Ser245, Thr251, Gly252, and Leu261.

The predicted high coupling efficiency sites were further screened to identify which of these sites retain PEG range of motion upon heterotrimerization with wild-type CD40L monomers. For PEG2000, sites for which greater than 20% of the simulated PEG chains are non-clashing in the heterotrimer state are preferred (see FIG. 3). These sites include Gly116, Asp117, Pro120, Ala130, Ser132, Lys133, Thr134, Lys143, Asn151, Asn157, Glu182, Ala183, Ser185, Gln186, Pro198, Gly199, Phe201, Pro217, Cyd218, Pro233, and Ser245. For PEG2000, sites for which greater than 30% of the simulated PEG are not clashing in the heterotrimer state are especially preferred. These sites include Gly116, Asp117, Ser132, Lys133, Thr134, Asn151, Gly199, Phe201, and Ser245. As discussed above, site specific PEGylation at any of these or other positions would either require replacement of the native amino acid with a suitable amino acid such as cysteine or the introduction of an unnatural amino acid such as p-acetyl-L-phenylalanine.

### EXAMPLE 3

Calculation of CD40L Substitutions Energies Using PDA® Algorithms.

In the present embodiment, the fitness of a substitution at a particular site in CD40L was judged by calculating the energy of all naturally occurring amino acids at the site. The crystal structure 1ALY.pdb was used as a starting model. The original PDB file is used as input into the algorithm REDUCE to generate a model with hydrogen atoms included and the side chains of asparagines, glutamines and histidines are adjusted by considering hydrogen bonding contacts that are frequently missed by crystallographers. The PDA® algorithms then consider every possible amino acid substitution at every position in the protein. The side chain orientation of each substitution is optimized for every amino acid fit into the site of interest. The best amino acid for the site is the amino acid that was used in the lowest energy rotamer at the site. Each rotamer's energy is judged by an energy function which uses Van der Waals, electrostatic, and hydrogen bonding terms as well as terms derived from statistical analyses of the current proteins in the Protein Data Bank. The relative weights, or strengths, of each term in determining the final energy of an amino acid at a site were determined by a training routine. This training routine choses the weights for each energy term based on predicting the wild-type amino acid, or a similar amino acid, to be the lowest energy amino acid at each position.

The PDA® calculations on CD40L are shown in FIG. 4 for all positions in the crystal structure 1ALY.pdb. For convenience, the lowest energy amino acid at each position is given an energy of zero and the other energies are adjusted accordingly. For example, at site 142 (FIG. 4b) the wild-type amino acid in human CD40L is E, glutamate. The PDA® calculations show that this amino acid is the most favorable at this position. Glutamine is the second most stable amino acid at position 142 having an energy of only 0.1kcal/mole. Therefore, glutamine is a suitable substitution at this position having an energy that is less than about 2 kcal/mole higher than the wild-type energy.

Phenylalanine 201, F201, is another example (FIG. 4d). In this case, the wild-type amino acid, phenylalanine, is an unfavorable amino acid at this position. In fact, Phe has the second highest energy of any amino acid allowed at this site in the PDA® calculations. This high energy for Phe in this position is due to its large exposure to solvent, which is unfavorable for a large, hydrophobic amino acid like Phe. At position 201, therefore, almost all of the mutations considered at this position are more stable than the wild-type amino acid. In this way, the PDA® algorithms can be used to identify positions that reduce the stability of the protein, which can lead to aggregation and generally unfavorable behavior physical properties.

All references cited herein including those cited above, including patents, patent applications (provisional, utility and PCT), and publications are incorporated by reference in their entirety.

Whereas particular embodiments of the invention have been described above for purposes of illustration, it will be appreciated by those skilled in the art that numerous variations of the details may be made without departing from the invention as described in the appended claims.

1. A composition comprising a variant CD40L protein having the formula:
Fx (45-115)-Vb (116)-Vb (117)-Vb(118)-Vb(119)-Vb(120)-Vb(121)-Fx(122)-Vb(123)-Fx(124)-Vb(125)-Fx(126)-Vb(127)-Vb(128)-Vb(129)-Vb(130)-Fx(131)-Vb(132)-Vb(133)-Vb(134)-Fx(135-136)-Vb(137)-Fx(138-139)-Vb(140)-Vb(141)-Vb(142)-Vb(143)-Vb(144)-Vb(145)-Vb(146)-Vb(147)-Vb(148)-Vb(149)-Fx(150)-Vb(151)-Vb(152)-Fx(153-154)-Vb(155)-Fx(156)-Vb(157)-Fx(158-165)-Vb(166)-Vb(167)-Vb(168)-Vb(169)-Vb(170)-Fx(171)-Vb(172)-Vb(173)-Vb(174)-Fx(175)-Vb(176)-Vb(177)-Vb(178)-Vb(179)-Fx(180)-Vb(181)-Vb(182)-Vb(183)-Vb(184)-Vb(185)-Vb(186)-Vb(187)-Vb(188)-Vb(189)-Vb(190)-Vb(191)-Vb(192)-Vb(193)-Vb(194)-Vb(195)-Vb(196)-Vb(197)-Vb(198)-Vb(199)-Vb(200)-Vb(201)-Vb(202)-Vb(203)-Vb(204)-Vb(205)-Vb(206)-Vb(207)-Vb(208)-Vb(209)-Vb(210)-Vb(211)-Fx(212)-Vb(213)-Vb(214)-Vb(215)-Vb(216)-Vb(217)-Vb(218)-Vb(219)-Vb(220)-Vb(221)-Vb(222)-Vb(223)-Vb(224)-Vb(225)-Vb(226)-Vb(227)-Vb(228)-Vb(229)-Vb(230)-Vb(231)-Vb(232)-Vb(233)-Fx(234-241)-Vb(242)-Vb(243)-Vb(244)-Vb(245)-Vb(246)-Vb(247)-Vb(248)-Vb(249)-Vb(250)-Vb(251)-Vb(252)-Vb(253)-Vb(254)-Vb(255)-Fx(256-258)-Vb(259)-Vb(260)-Vb(261)
wherein
Fx(45-115) is optionally present, and if present, comprises the human amino acid sequence of CD40L at positions 45-115;
Vb(116) is selected from the group consisting of G and C;
Vb(117) is selected from the group consisting of D and C;
Vb(118) is selected from the group consisting of Q, C, H, E, N, A, T, S, W, F, V, G, K, R, Y, D, and M;
Vb(119) is selected from the group consisting of N, H, Q, L, N, R, D, A, S, M, K, G, and E;
Vb(120) is selected from the group consisting of P, C, R, Q, N, E, D, K, G, and S;
Vb(121) is selected from the group consisting of Q, H, T, V, Y, M, C, D, A, E, I, N, F, L, S, and G;
Fx(122) comprises the human amino acid sequence of CD40L at position 122;
Vb(123) is selected from the group consisting of A and E;
Fx(124) comprises the human amino acid sequence of CD40L at position 124;
Vb(125) is selected from the group consisting of H, R, N, and M;
Fx(126) is selected from the group consisting of V, D, and A;
Vb(127) is selected from the group consisting of H, Q, E, C, S, D, and I;
Vb(128) is selected from the group consisting of S and R;
Vb(129) is selected from the group consisting of E and G;
Vb(130) is selected from the group consisting of P, Q, K, M, N, C, R, E, D, and A;
Fx(131) comprises the human amino acid sequence of CD40L at position 131;
Vb(132) is selected from the group consisting of C and S;
Vb(133) is selected from the group consisting of C and K;
Vb(134) is selected from the group consisting of C, Q, E, D, R, N, K, S, G, and T;
Fx(135-136) comprises the human amino acid sequence of CD40L at positions 135-136;
Vb(137) is selected from the group consisting of R, K, S, and V;
Fx(138-139) comprises the human amino acid sequence of CD40L at positions 138-139;
Vb(140) is selected from the group consisting of W, C, G, R, and a deletion;
Vb(141) comprises any amino acid;
Vb(142) is selected from the group consisting of M, V, C, I, E, and a deletion;
Vb(143) is selected from the group consisting of C, E, K, and a deletion;
Vb(144) is selected from the group consisting of Q, M, E, H, K, R, and G;
Vb(145) is selected from the group consisting of P, E, N, C, D, H, Y, and a deletion;
Vb(146) is selected from the group consisting of S, C, and N;
Vb(147) is selected from the group consisting of T, V, I, and N;
Vb(148) is selected from the group consisting of M and K;
Vb(149) is selected from the group consisting of S and A;
Fx(150) comprises the human amino acid sequence of CD40L at position 150;
Vb(151) comprises any amino acid;
Vb(152) is selected from the group consisting of L, E, and Q;
Fx(153-154) comprises the human amino acid sequence of CD40L at positions 153-154;
Vb(155) is selected from the group consisting of L and P;
Fx(156) comprises the human amino acid sequence of CD40L at position 156;
Vb(157) comprises any amino acid;
Fx(158-165) comprises the human amino acid sequence of CD40L at positions 158-165;
Vb(166) is selected from the group consisting of Q, S, G, C, D, Q, E, A, and N;
Vb(167) comprises any amino acid;
Vb(168) is selected from the group consisting of C, P, and L;
Vb(169) is selected from the group consisting of Y and D;
Vb(170) is selected from the group consisting of Y, C, and P;
Fx(171) comprises the human amino acid sequence of CD40L at position 171;
Vb(172) comprises any amino acid;
Vb(173) is selected from the group consisting of A and D;
Vb(174) is selected from the group consisting of Q, R, I, V, L, H, and K;
Fx(175) comprises the human amino acid sequence of CD40L at position 175;
Vb(176) is selected from the group consisting of T, G, I, A, S, and G;
Vb(177) comprises any amino acid;
Vb(178) is selected from the group consisting of C, G, S, D, E, H, Q, N, K, Y, M, and R;
Vb(179) comprises any amino acid;
Fx(180) comprises the human amino acid sequence of CD40L at position 180;
Vb(181) is selected from the group consisting of R, G, S, C, and A;
Vb(182) is selected from the group consisting of E and C;
Vb(183) is selected from the group consisting of A, Q, E, R, D, N, K, S, G, and an insertion;
Vb(184) is selected from the group consisting of S, C, L, R, M, and deletion;
Vb(185) is selected from the group consisting of S, R, H, N, K, H, T, A, and Q;
Vb(186) is selected from the group consisting of Q, T, I, V, L, A, C, H, K, R, N, M, E, D, S, and deletion;
Vb(187) is selected from the group consisting of A, E, H, W, Q, M, D, L, R, N, and K;
Vb(188) is selected from the group consisting of P, D, N, H, E, M, C, Q, K, T, A, V, S, and G;
Vb(189) comprises any amino acid;
Vb(190) is selected from the group consisting of I, Y, A, C, S, E, and V;
Vb(191) comprises any amino acid;
Vb(192) comprises any amino acid;
Vb(193) is selected from the group consisting of L and an insertion;
Vb(194) is selected from the group consisting of C, A, Q, K, and M;
Vb(195) is selected from the group consisting of L and P;
Vb(196) comprises any amino acid;
Vb(197) comprises any amino acid;
Vb(198) is selected from the group consisting of C, E, D, N, Q, and P;
Vb(199) comprises any amino acid;
Vb(200) is selected from the group consisting of R, D, M, S, Q, H, N, and a deletion;
Vb(201) is selected from the group consisting of F, R, H, E, N, Q, D, K, S, T, and G;
Vb(202) is selected from the group consisting of E and a deletion;
Vb(203) is selected from the group consisting of R, Y, F, T, W, A, S, D, C, I, E, N, H, and Q;
Vb(204) is selected from the group consisting of I, E, Q, S, V, P, L, M, T, Q, and C;
Vb(205) comprises any amino acid;
Vb(206) is selected from the group consisting of L, Q, M, and E;
Vb(207) is selected from the group consisting of R, C, E, and N;
Vb(208) is selected from the group consisting of A and D;
Vb(209) is selected from the group consisting of C, S, and A;
Vb(210) is selected from the group consisting of N, Q, C, and E;
Vb(211) is selected from the group consisting of C, D, Y, and E;
Fx(212) comprises the human amino acid sequence of CD40L at position 212;
Vb(213) is selected from the group consisting of S, D, and A;
Vb(214) is selected from the group consisting of S, P, R, D, N, A, E, and Q;
Vb(215) is selected from the group consisting of C, G, A, and S;
Vb(216) is selected from the group consisting of K and D;
Vb(217) is selected from the group consisting of P, D, E, Q, S, N, G, Y, H, F, and A;
Vb(218) is selected from the group consisting of C, P, D, G, and a deletion;
Vb(219) comprises any amino acid;
Vb(220) is selected from the group consisting of Q, P, C, T, E, D, M, N, and a deletion;
Vb(221) is selected from the group consisting of Q, L, Y, M, E, S, D, F, and a deletion;
Vb(222) is selected from the group consisting of S, A, T, and C;
Vb(223) is selected from the group consisting of P, E, D, S, Q, N, R, and G;
Vb(224) is selected from the group consisting of H, Y, and F;
Vb(225) comprises any amino acid;
Vb(226) is selected from the group consisting of G and A;
Vb(227) is selected from the group consisting of G and V;
Vb(228) comprises any amino acid;
Vb(229) comprises any amino acid;
Vb(230) is selected from the group consisting of E, P, H, M, and Q;
Vb(231) is selected from the group consisting of L and S;
Vb(232) is selected from the group consisting of Q, D, E, H, N, Y, V, S, and a deletion;
Vb(233) comprises any amino acid;
Fx(234-241) comprises the human amino acid sequence of CD40L at positions 234-241;
Vb(242) comprises any amino acid;
Vb(243) comprises any amino acid;
Vb(244) comprises any amino acid;
Vb(245) is selected from the group consisting of S, R, P, H, M, V, L, Y, and I;
Vb(246) comprises any amino acid;
Vb(247) comprises any amino acid;
Vb(248) is selected from the group consisting of S and C;
Vb(249) is selected from the group consisting of H and D;
Vb(250) comprises any amino acid;
Vb(251) is selected from the group consisting of T, E, Q, M, D, P, H, I, L, R, V, N, A, S, and G;
Vb(252) comprises any amino acid;
Vb(253) is selected from the group consisting of F, E, D, H, Y, and M;
Vb(254) is selected from the group consisting of T and M;
Vb(255) is selected from the group consisting of S, U, C, T, and A;
Fx(256-258) comprises the human amino acid sequence of CD40L at positions 256-258;
Vb(259) comprises any amino acid;
Vb(260) is selected from the group consisting of K, S, A, C, Q, M, N, and E;
Vb(261) is selected from the group consisting of L, N, S, D, V, I, T, C, and A.
wherein at least one of said amino acids at at least one of said positions is the not the wild-type amino acid.

2. A composition according to claim 1 wherein said variant comprises a polymer.

3. A composition according to claim 2 wherein said polymer comprises polyethylene glycol (PEG).

4. A composition according to claim 3 wherein said PEG is attached at an amino acid position selected from the group consisting of 116, 117, 118, 120, 121, 130, 132, 133, 134, 143, 145, 151, 157, 168, 182, 183, 185, 186, 187, 198, 199, 201, 203, 209, 211, 213, 215, 217, 218, 220, 224, 228, 230, 233, 245, 251, 252 and 261.

5. A composition according to claims 1 to 4 wherein said composition comprises a TNF-a trimer.

6. A composition according to claim 5 wherein said trimer comprises three variant monomers according to said formula.

7. A composition according to claim 5 wherein said trimer comprises three identical variant monomers.

8. A composition according to claim 5 wherein said trimer comprises at least one variant monomer and at least one wild-type monomer.

9. A composition according to claims 1 to 4 comprising two of said variant monomers covalently attached via a linker.

10. A composition according to claim 9 wherein said linker is a polypeptide linker.

11. A composition according to claim 10 wherein said polypeptide linker is attached to the N-terminus of a first monomer and the C-terminus of a second monomer.

12. A composition according to claim 1 wherein Vb positions 141, 151, 157, 167, 172, 177, 179, 189, 191, 192, 196, 197, 199, 205, 219, 225, 228, 229, 233, 242, 243, 244, 246, 247, 250, 252 and 259 are human wild-type amino acids.

13. A composition according to claim 1 further comprising a pharmaceutical carrier.

14. A method of treating a CD40L related disorder comprising administering to said patient a composition according to claim 1.

15. A method according to claim 14 wherein said composition comprises a CD40L trimer.

16. A method according to claim 15 wherein said trimer comprises three variant monomers according to said formula.

17. A method according to claim 16 wherein said trimer comprises three identical variant monomers.

18. A method according to claim 15 wherein said trimer comprises at least one variant monomer and at least one wild-type monomer.

19. A method according to claim 14 comprising two of said variant monomers covalently attached via a linker.

20. A method according to claim 14 wherein said variant comprises at least one PEG molecule.

21. A composition comprising a PEGylated variant CD40L protein having the formula:
Fx (45-115)-Vb (116)-Vb (117)-Vb(118)-Vb(119)-Vb(120)-Vb(121)-Fx(122)-Vb(123)-Fx(124)-Vb(125)-Fx(126)-Vb(127)-Vb(128)-Vb(129)-Vb(130)-Fx(131)-Vb(132)-Vb(133)-Vb(134)-Fx(135-136)-Vb(137)-Fx(138-139)-Vb(140)-Vb(141)-Vb(142)-Vb(143)-Vb(144)-Vb(145)-Vb(146)-Vb(147)-Vb(148)-Vb(149)-Fx(150)-Vb(151)-Vb(152)-Fx(153-154)-Vb(155)-Fx(156)-Vb(157)-Fx(158-165)-Vb(166)-Vb(167)-Vb(168)-Vb(169)-Vb(170)-Fx(171)-Vb(172)-Vb(173)-Vb(174)-Fx(175)-Vb(176)-Vb(177)-Vb(178)-Vb(179)-Fx(180)-Vb(181)-Vb(182)-Vb(183)-Vb(184)-Vb(185)-Vb(186)-Vb(187)-Vb(188)-Vb(189)-Vb(190)-Vb(191)-Vb(192)-Vb(193)-Vb(194)-Vb(195)-Vb(196)-Vb(197)-Vb(198)-Vb(199)-Vb(200)-Vb(201)-Vb(202)-Vb(203)-Vb(204)-Vb(205)-Vb(206)-Vb(207)-Vb(208)-Vb(209)-Vb(210)-Vb(211)-Fx(212)-Vb(213)-Vb(214)-Vb(215)-Vb(216)-Vb(217)-Vb(218)-Vb(219)-Vb(220)-Vb(221)-Vb(222)-Vb(223)-Vb(224)-Vb(225)-Vb(226)-Vb(227)-Vb(228)-Vb(229)-Vb(230)-Vb(231)-Vb(232)-Vb(233)-Fx(234-241)-Vb(242)-Vb(243)-Vb(244)-Vb(245)-Vb(246)-Vb(247)-Vb(248)-Vb(249)-Vb(250)-Vb(251)-Vb(252)-Vb(253)-Vb(254)-Vb(255)-Fx(256-258)-Vb(259)-Vb(260)-Vb(261)
wherein
Fx(45-115) is optionally present, and if present, comprises the human amino acid sequence of CD40L at positions 45-115;
Vb(116) is selected from the group consisting of G and C;
Vb(117) is selected from the group consisting of D and C;
Vb(118) is selected from the group consisting of Q, C, H, E, N, A, T, S, W, F, V, G, K, R, Y, D, and M;
Vb(119) is selected from the group consisting of N, H, Q, L, N, R, D, A, S, M, K, G, and E;
Vb(120) is selected from the group consisting of P, C, R, Q, N, E, D, K, G, and S;
Vb(121) is selected from the group consisting of Q, H, T, V, Y, M, C, D, A, E, I, N, F, L, S, and G;
Fx(122) comprises the human amino acid sequence of CD40L at position 122;
Vb(123) is selected from the group consisting of A and E;
Fx(124) comprises the human amino acid sequence of CD40L at position 124;
Vb(125) is selected from the group consisting of H, R, N, and M;
Fx(126) is selected from the group consisting of V, D, and A;
Vb(127) is selected from the group consisting of H, Q, E, C, S, D, and I;
Vb(128) is selected from the group consisting of S and R;
Vb(129) is selected from the group consisting of E and G;
Vb(130) is selected from the group consisting of P, Q, K, M, N, C, R, E, D, and A;
Fx(131) comprises the human amino acid sequence of CD40L at position 131;
Vb(132) is selected from the group consisting of C and S;
Vb(133) is selected from the group consisting of C and K;
Vb(134) is selected from the group consisting of C, Q, E, D, R, N, K, S, G, and T;
Fx(135-136) comprises the human amino acid sequence of CD40L at positions 135-136;
Vb(137) is selected from the group consisting of R, K, S, and V;
Fx(138-139) comprises the human amino acid sequence of CD40L at positions 138-139;
Vb(140) is selected from the group consisting of W, C, G, R, and a deletion;
Vb(141) comprises any amino acid;
Vb(142) is selected from the group consisting of M, V, C, I, E, and a deletion;
Vb(143) is selected from the group consisting of C, E, K, and a deletion;
Vb(144) is selected from the group consisting of Q, M, E, H, K, R, and G;
Vb(145) is selected from the group consisting of P, E, N, C, D, H, Y, and a deletion;
Vb(146) is selected from the group consisting of S, C, and N;
Vb(147) is selected from the group consisting of T, V, I, and N;
Vb(148) is selected from the group consisting of M and K;
Vb(149) is selected from the group consisting of S and A;
Fx(150) comprises the human amino acid sequence of CD40L at position 150;
Vb(151) comprises any amino acid;
Vb(152) is selected from the group consisting of L, E, and Q;
Fx(153-154) comprises the human amino acid sequence of CD40L at positions 153-154;
Vb(155) is selected from the group consisting of L and P;
Fx(156) comprises the human amino acid sequence of CD40L at position 156;
Vb(157) comprises any amino acid;
Fx(158-165) comprises the human amino acid sequence of CD40L at positions 158-165;
Vb(166) is selected from the group consisting of Q, S, G, C, D, Q, E, A, and N;
Vb(167) comprises any amino acid;
Vb(168) is selected from the group consisting of C, P, and L;
Vb(169) is selected from the group consisting of Y and D;
Vb(170) is selected from the group consisting of Y, C, and P;
Fx(171) comprises the human amino acid sequence of CD40L at position 171;
Vb(172) comprises any amino acid;
Vb(173) is selected from the group consisting of A and D;
Vb(174) is selected from the group consisting of Q, R, I, V, L, H, and K;
Fx(175) comprises the human amino acid sequence of CD40L at position 175;
Vb(176) is selected from the group consisting of T, G, I, A, S, and G;
Vb(177) comprises any amino acid;
Vb(178) is selected from the group consisting of C, G, S, D, E, H, Q, N, K, Y, M, and R;
Vb(179) comprises any amino acid;
Fx(180) comprises the human amino acid sequence of CD40L at position 180;
Vb(181) is selected from the group consisting of R, G, S, C, and A;
Vb(182) is selected from the group consisting of E and C;
Vb(183) is selected from the group consisting of A, Q, E, R, D, N, K, S, G, and an insertion;
Vb(184) is selected from the group consisting of S, C, L, R, M, and deletion;
Vb(185) is selected from the group consisting of S, R, H, N, K, H, T, A, and Q;
Vb(186) is selected from the group consisting of Q, T, I, V, L, A, C, H, K, R, N, M, E, D, S, and deletion;
Vb(187) is selected from the group consisting of A, E, H, W, Q, M, D, L, R, N, and K;
Vb(188) is selected from the group consisting of P, D, N, H, E, M, C, Q, K, T, A, V, S, and G;
Vb(189) comprises any amino acid;
Vb(190) is selected from the group consisting of I, Y, A, C, S, E, and V;
Vb(191) comprises any amino acid;
Vb(192) comprises any amino acid;
Vb(193) is selected from the group consisting of L and an insertion;
Vb(194) is selected from the group consisting of C, A, Q, K, and M;
Vb(195) is selected from the group consisting of L and P;
Vb(196) comprises any amino acid;
Vb(197) comprises any amino acid;
Vb(198) is selected from the group consisting of C, E, D, N, Q, and P;
Vb(199) comprises any amino acid;
Vb(200) is selected from the group consisting of R, D, M, S, Q, H, N, and a deletion;
Vb(201) is selected from the group consisting of F, R, H, E, N, Q, D, K, S, T, and G;
Vb(202) is selected from the group consisting of E and a deletion;
Vb(203) is selected from the group consisting of R, Y, F, T, W, A, S, D, C, I, E, N, H, and Q;
Vb(204) is selected from the group consisting of I, E, Q, S, V, P, L, M, T, Q, and C;
Vb(205) comprises any amino acid;
Vb(206) is selected from the group consisting of L, Q, M, and E;
Vb(207) is selected from the group consisting of R, C, E, and N;
Vb(208) is selected from the group consisting of A and D;
Vb(209) is selected from the group consisting of C, S, and A;
Vb(210) is selected from the group consisting of N, Q, C, and E;
Vb(211) is selected from the group consisting of C, D, Y, and E;
Fx(212) comprises the human amino acid sequence of CD40L at position 212;
Vb(213) is selected from the group consisting of S, D, and A;
Vb(214) is selected from the group consisting of S, P, R, D, N, A, E, and Q;
Vb(215) is selected from the group consisting of C, G, A, and S;
Vb(216) is selected from the group consisting of K and D;
Vb(217) is selected from the group consisting of P, D, E, Q, S, N, G, Y, H, F, and A;
Vb(218) is selected from the group consisting of C, P, D, G, and a deletion;
Vb(219) comprises any amino acid;
Vb(220) is selected from the group consisting of Q, P, C, T, E, D, M, N, and a deletion;
Vb(221) is selected from the group consisting of Q, L, Y, M, E, S, D, F, and a deletion;
Vb(222) is selected from the group consisting of S, A, T, and C;
Vb(223) is selected from the group consisting of P, E, D, S, Q, N, R, and G;
Vb(224) is selected from the group consisting of H, Y, and F;
Vb(225) comprises any amino acid;
Vb(226) is selected from the group consisting of G and A;
Vb(227) is selected from the group consisting of G and V;
Vb(228) comprises any amino acid;
Vb(229) comprises any amino acid;
Vb(230) is selected from the group consisting of E, P, H, M, and Q;
Vb(231) is selected from the group consisting of L and S;
Vb(232) is selected from the group consisting of Q, D, E, H, N, Y, V, S, and a deletion;
Vb(233) comprises any amino acid;
Fx(234-241) comprises the human amino acid sequence of CD40L at positions 234-241;
Vb(242) comprises any amino acid;
Vb(243) comprises any amino acid;
Vb(244) comprises any amino acid;
Vb(245) is selected from the group consisting of S, R, P, H, M, V, L, Y, and I;
Vb(246) comprises any amino acid;
Vb(247) comprises any amino acid;
Vb(248) is selected from the group consisting of S and C;
Vb(249) is selected from the group consisting of H and D;
Vb(250) comprises any amino acid;
Vb(251) is selected from the group consisting of T, E, Q, M, D, P, H, I, L, R, V, N, A, S, and G;
Vb(252) comprises any amino acid;
Vb(253) is selected from the group consisting of F, E, D, H, Y, and M;
Vb(254) is selected from the group consisting of T and M;
Vb(255) is selected from the group consisting of S, U, C, T, and A;
Fx(256-258) comprises the human amino acid sequence of CD40L at positions 256-258;
Vb(259) comprises any amino acid;
Vb(260) is selected from the group consisting of K, S, A, C, Q, M, N, and E;
Vb(261) is selected from the group consisting of L, N, S, D, V, I, T, C, and A.
wherein at least one PEG molecule is attached at an amino acid position selected from the group consisting of 116, 117, 118, 120, 121, 130, 132, 133, 134, 143, 145, 151, 157, 168, 182, 183, 185, 186, 187, 198, 199, 201, 203, 209, 211, 213, 215, 217, 218, 220, 224, 228, 230, 233, 245, 251, 252 and 261.

22. A method of making a PEGylated CD40L molecule comprising:
a) substituting a cysteine at at least one position selected from the group consisting of 116, 117, 118, 120, 121, 130, 132, 133, 134, 143, 145, 151, 157, 168, 182, 183, 185, 186, 187, 198, 199, 201, 203, 209, 211, 213, 215, 217, 218, 220, 224, 228, 230, 233, 245, 251, 252 and 261; and
b) adding a PEG molecule to said position.

23. A composition according to claim 6 wherein said trimer comprises three identical variant monomers.
